(19) United States
(12) Patent Application Publication
(10) Pub. No.: US 2022/0403397 A1
(43) Pub. Date: Dec. 22, 2022

(54) NUCLEIC ACID CONSTRUCTS ENCODING KALLIKREIN-2 FUSION PROTEIN AND VECTORS, PREPARATIONS OF CELLS, AND METHODS OF USE THEREOF

(71) Applicant: Janssen Biotech, Inc., Horsham, PA (US)

(72) Inventors: Stuart L. EMANUEL, Doylestown, PA (US); Thomas J. RUTKOSKI, Philadelphia, PA (US)

(21) Appl. No.: 17/835,671

(22) Filed: Jun. 8, 2022

Related U.S. Application Data
(60) Provisional application No. 63/209,019, filed on Jun. 10, 2021.

(51) Int. Cl. C12N 15/62; C12N 15/86; C12N 15/87; A61P 35/00; C12N 9/64

(52) U.S. Cl. CPC C12N 15/62; C12N 15/86; C12N 15/87; A61P 35/00; C12N 9/6445

(57) ABSTRACT

The present invention relates to a recombinant nucleic acid construct encoding a kallikrein-2 fusion protein. The kallikrein-2 fusion protein includes a first nucleotide sequence encoding kallikrein-2 (KLK2), and a second nucleotide sequence encoding a glycosylphophatidylinositol (GPI) attachment sequence, where the GPI attachment sequence encoding nucleotide sequence is positioned 3′ to the KLK2 encoding nucleotide sequence. Also disclosed are vectors, preparations of cells, and methods of use thereof

### CROSS-REFERENCE TO RELATED APPLICATION

This application claims priority to U.S. Provisional Application Ser. No. 63/209,019, filed 10 Jun. 2021, the entire contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 21, 2022, is named JBI6578WOPCT1_SL.txt and is 33,873 bytes in size.

### FIELD

The present invention relates to nucleic acid constructs encoding kallikrein-2 fusion proteins, as well as vectors, preparations of cells, and methods of use thereof.

### BACKGROUND

The family of human Kallikreins (KLKs) is comprised of 15 serine proteases with diverse biological functions and tissue distribution (Thorek et al., Thromb. Haemost. 110(30):4840-92 (2013)). Kallikrein-2 (KLK2) is highly and selectively expressed in normal prostate, primary prostate cancer, and metastatic castration-resistant prostate cancer. Its expression is regulated by androgens and closely correlated to androgen receptor expression. Its tissue specificity makes it an attractive target for therapies targeting prostate cancer. However, KLK2 (also referred to as hK2, UniProt P20151) is a secreted protein in its catalytically active that is often attached to the prostate tumor cell surface through unknown mechanisms. It is highly and selectively expressed in normal prostate, primary prostate cancer, and metastatic castration-resistant prostate cancer, making it an attractive target for therapies targeting prostate cancer. Commercially available prostate tumor cells expressing endogenous KLK2 on the cell surface are limited. VCaP and LNCaP prostate tumor cell lines express detectable cell surface KLK2, albeit at very low levels compared to primary tumor cells. The lack of appropriate tumor cell lines makes it difficult to identify and validate potential therapeutics that intervene with the KLK2 pathway.

Past attempts have been made to overexpress KLK2 in KLK2-negative prostate tumor cell lines DU145 and PC3 as well as in many other cell lines. However, they have all failed to produce tumor cell lines with KLK2 surface expression because the KLK2 protein was either expressed intracellularly or secreted to the extracellular matrix (e.g., CHO-K1, HEK293, NS0, LnCap).

The present invention is directed to overcoming these and other deficiencies in the art.

### SUMMARY

A first aspect of the present disclosure is directed to a recombinant nucleic acid construct encoding a kallikrein-2 fusion protein. The recombinant nucleic acid construct comprises a first nucleotide sequence encoding kallikrein-2 (KLK2) and a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

Another aspect of the present disclosure is directed to a preparation of cells, where cells of the preparation express, on their surface, a recombinant kallikrein-2 fusion protein. The fusion protein includes a kallikrein-2 polypeptide sequence, a portion of a glycosylphophatidylinositol (GPI) attachment sequence linked to the C-terminus of the kallikrein-2 polypeptide sequence; and a GPI anchor domain coupled to the GPI attachment sequence portion.

A further aspect of the present disclosure is directed to a non-human animal comprising cells expressing, on their surface, a recombinant kallikrein-2 fusion protein. The recombinant fusion protein includes a kallikrein-2 polypeptide sequence; a portion of a glycosylphophatidylinositol (GPI) attachment sequence linked to the C-terminus of the kallikrein-2 polypeptide sequence; and a GPI anchor domain coupled to the GPI attachment sequence portion.

Yet a further aspect of the present disclosure is directed to a method of identifying an agent that binds kallikrein-2. This method involves providing a preparation of cells according to the present disclosure; administering a candidate agent to the preparation of cells; and determining whether the candidate agent binds kallikrein-2 based on said administering.

Another aspect of the present disclosure is directed to a method of identifying an agent that binds kallikrein-2. This method involves providing a non-human animal according to the present disclosure; administering a candidate agent to the non-human animal; and determining whether the candidate agent binds kallikrein-2 based on said administering.

The disclosure comprises a method for engineering surface expression of kallikrein-2 in cells by creating a kallikrein-2 fusion protein with the glycosylphosphatidylinositol (GPI) attachment sequence of human placental alkaline phosphatase (PLAP). Expression of the protein within the transfected cells is driven by the EF1α promoter, and the kallikrein-2 fusion protein is anchored to the cell membrane by a GPI anchoring domain coupled to the GPI attachment sequence. This method is useful for achieving surface expression in cells that do not express kallikrein-2 or overexpression in cells that express endogenous kallikrein-2. Conventional methods of expressing kallikrein-2 have failed to display KLK2 on the cell surface, producing only intracellular or extracellular expression, or no expression at all. Cells with KLK2 engineered on the surface have utility for screening and identifying KLK2 therapeutics (e.g., cell therapy products, CD3 redirecting antibodies, antibody-dependent cellular cytotoxicity (ADCC)-mediated antibodies, etc.) in release assays or in in vitro or in vivo experimental systems.

### BRIEF DESCRIPTION OF THE DRAWINGS

FIG. 1 is a histogram showing KLK2 surface expression in DU145 cells transduced with the KLK2-GPI fusion construct (“KLK2_GPI”) as described herein. Cells were stained with isotype control or anti-KLK2 clone KL2B1 directly conjugated to PE.

FIGS. 2A-2C are graphs showing the binding of hIgG1 isotype control Ab or anti-KLK2-specific Abs on VCaP (FIG. 2A), DU145 parental cells (FIG. 2B), or DU145/KLK2_GPI tumor cells (FIG. 2C).

FIGS. 3A-3C are graphs showing the binding of hIgG1 isotype control Ab or anti-KLK2-specific Abs on PC3 parental cells (FIG. 3A), PC3/KLK2_GPI (FIG. 3B), or PC3/PSMA/KLK2_GPI tumor cells (FIG. 3C).

FIGS. 4A-4C are graphs showing antibody-dependent cellular cytotoxicity (ADCC) against VCaP (FIG. 4A), DU145 parental cells (FIG. 4B), or DU145/KLK2_GPI tumor cells (FIG. 4C). PB-NK cells were co-cultured with tumor cells at an E:T ratio of 3:1. The number of live tumor target cells were counted after 66 hours using IncuCyte. The number of live tumor targets remaining at the end of assay were normalized to tumor only wells to generate % live tumor targets.

FIGS. 5A-5B are graphs showing ADCC against PC3 parental cells (FIG. 5A) or PC3/PSMA/KLK2_GPI tumor cells (FIG. 5B). PB-NK cells were co-cultured with tumor cells at an Effector:Tumor (E:T) ratio of 3:1 in the presence of anti-KLK2 antibodies or isotype control antibody. The number of live tumor target cells were counted after 66 hours using IncuCyte. The number of live tumor targets remaining at the end of assay were normalized to tumor only wells to generate % live tumor targets.

FIG. 6 is a graph showing the cytotoxicity of KLK2×CD3 bispecific antibody against VCaP, LnCap/KLK2, or DU145/KLK2_GPI tumor cells. Primary T cells were co-cultured with tumor cells at an E:T ratio of 3:1 in the presence of anti-KLK2 antibodies or isotype control antibody. Increasing concentration of KLK2×CD3 bispecific Abs were mixed with tumor cells and T cells. The number of live tumor target cells were counted after 72 hours using IncuCyte. The number of live tumor targets that remained at the end of assay were normalized to tumor only wells to generate % Tumor Lysis.

FIGS. 7A-7C are graphs showing CAR-T-mediated cytotoxicity against VCaP (FIG. 7A), parental DU145 (FIG. 7B), or DU145/KLK2_GPI tumor cells (FIG. 7C). Untransduced (UTD) T cells or KLK2 CAR-transduced T cells were co-cultured with tumor cells at an E:T ratio of 0.25:1. The number of live tumor target cells were counted every 24 hours starting at time 0 using IncuCyte. The number of live tumor targets that remained at each timepoint were normalized to tumor only wells to generate % Tumor Live Tumor Targets.

FIGS. 8A-8B are graphs showing application of DU145/KLK2_GPI and PC3/PSMA/KLK2_GPI tumor cells in vivo. (FIG. 8A) Growth kinetics of DU145/KLK2_GPI and PC3/PSMA/KLK2_GPI. 10×10⁶ DU145/KLK2_GPI tumor cells or 0.5×10⁶ PC3/PSMA/KLK2_GPI tumor cells were implanted on day 0. Tumors were measured by caliper every 3 or 4 days. (FIG. 8B) Efficacy of anti-KLK2 CART cells in DU145/KLK2_GPI tumor model. 10×10⁶ KLK2 CART cells were injected on day 11 post-tumor implantation. Tumors were measured by caliper every 3 or 4 days. KLK2 CAR T cells inhibited tumor progression and caused complete tumor regression.

FIGS. 9A-9C show how DU145+KLK2 cells can be used to screen CAR designs. A panel of CAR designs (CAR-a to CAR-bb) were transduced into NK-101 cells. These designs all contained the same scFv binding domain specific for KLK2 followed by the CD8a hinge region and various different signaling domain modules.

FIGS. 10A-10B show a histogram demonstrating KLK2 surface expression in LnCap cells transduced with the KLK2-GPI fusion construct (“KLK2_GPI”) as described herein. Cells were stained with isotype control or anti-KLK2 clone KL2B1 directly conjugated to PE.

FIG. 10C are graphs showing KLK2 CAR-NK-mediated cytotoxicity against LnCap parent (untransduced) cells or LnCap+KLK2 target cells that were co-cultured at various E:T ratios. The number of live tumor target cells were counted every 4 hours starting at time 0 using IncuCyte. The number of live tumor targets that remained at each timepoint were normalized to tumor only wells to generate % Live Tumor Targets remaining. The AUC of the % Live Tumor Target curve over 166 hours was determined for each E:T ratio and plotted as a dose-response curve. The innate or non-CAR-specific killing can be determined from LnCap parent cells while the KLK2 CAR-specific killing can be assessed in the LnCap+KLK2 target cells.

### DETAILED DESCRIPTION

A first aspect of the present disclosure is directed to a recombinant nucleic acid construct encoding a kallikrein-2 fusion protein. The recombinant nucleic acid construct comprises a first nucleotide sequence encoding kallikrein-2 (KLK2) or a fragment thereof and a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

The first nucleotide sequence of the recombinant construct encoding kallikrein-2 may encode a mammalian kallikrein-2 polypeptide sequence, e.g., a human, murine, bovine, canine, feline, ovine, porcine, ursine, or simian kallikrein-2 polypeptide sequence.

In any embodiment, the first nucleotide sequence encoding kallikrein-2 of the recombinant construct encodes a human kallikrein-2 (hKLK2). As described herein, human Kallikrein-2 (“hKLK2” or “hK2”) is a prostate-specific kallikrein (see, e.g., Obiezu et al., “Human Tissue Kallikrein Gene Family: Applications in Cancer,” Cancer Letters 224(1):1-22 (2005) and Nasser et al., “Human Tissue Kallikreins: Blood Levels and Response to Radiotherapy in Intermediate Risk Prostate Cancer,” Radiother. Oncol. 124(3):427-432 (2017), which are hereby incorporated by reference in their entirety).

In any embodiment, the first nucleotide sequence encodes a human kallikrein-2 comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO:4 or a functional fragment thereof.

(SEQ ID NO: 4)
MWDLVLSIALSVGCTGAVPLIQSRIVGGWECEKHSQPWOVAVYSHGWAH
CGGVLVHPQWVLTAAHCLKKNSQVWLGRHNLFEPEDTGQRVPVSHSFPH
PLYNMSLLKHQSLRPDEDSSHDLMLLRLSEPAKITDVVKVLGLPTQEPA
LGTTCYASGWGSIEPEEFLRPRSLQCVSLHLLSNDMCARAYSEKVTEFM
LCAGLWTGGKDTCGGDSGGPLVCNGVLQGITSWGPEPCALPEKPAVYTK
VVHYRKWIKDTIAANPEF (signal sequence shown in
double underline).

In any embodiment, the first nucleotide sequence encodes a human kallikrein-2 comprising an amino acid sequence of SEQ ID NO: 4 or a functional fragment thereof.

In any embodiment, the first nucleotide sequence encoding kallikrein-2 comprises a nucleotide sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO:1 or any portion thereof.

(SEQ ID NO: 1)
ATGTGGGACCTGGTTCTCTCCATCGCCTTGTCTGTGGGGTGCACTGGTG
CCGTGCCCCTCATCCAGTCTCGGATCGTGGGGGGCTGGGAGTGCGAGAA
GCACAGCCAGCCTTGGCAAGTGGCAGTGTACTCCCACGGTTGGGCGCAC
TGCGGTGGCGTGCTGGTGCACCCACAATGGGTGCTCACCGCGGCCCACT
GTCTGAAGAAGAATTCACAAGTCTGGCTGGGACGCCATAACCTGTTCGA
ACCTGAAGATACTGGGCAGCGCGTGCCGGTGTCCCATTCCTTCCCTCAC
CCATTGTACAACATGTCGCTGCTGAAGCACCAGTCTTTGAGGCCTGATG
AGGACAGCTCCCATGACCTCATGCTGCTTAGACTCTCGGAACCCGCAAA
GATTACCGACGTCGTGAAAGTGCTTGGACTGCCGACGCAGGAACCCGCC
TTGGGGACTACCTGTTATGCTTCCGGCTGGGGATCCATCGAGCCCGAAG
AATTCCTGCGGCCGCGCAGCCTGCAGTGCGTGTCCCTCCATCTGCTGTC
AAACGATATGTGCGCCAGAGCCTACTCCGAAAAGGTCACCGAGTTTATG
CTGTGCGCCGGACTGTGGACCGGGGGAAAGGACACTTGCGGCGGAGACA
GCGGCGGCCCCCTGGTCTGCAACGGCGTGCTGCAGGGAATTACCTCGTG
GGGTCCAGAGCCGTGTGCGCTGCCTGAAAAGCCCGCCGTGTACACTAAG
GTCGTGCACTACCGGAAGTGGATCAAGGACACCATCGCCGCGAACCCGG
AATTC

The nucleotide sequence encoding the signal sequence of kallikrein-2 is double underlined in SEQ ID NO: 1. Thus, in any embodiment, the nucleotide sequence encoding kallikrein-2 comprises the nucleotide sequence of SEQ ID NO:1. In any embodiment, the nucleotide sequence encoding kallikrein-2 comprises the nucleotide sequence of SEQ ID NO: 1 without the signal sequence. In any embodiment, the nucleotide sequence encoding kallikrein-2 comprises a portion or fragment of the nucleotide sequence of SEQ ID NO: 1.

Glycosylphosphatidylinositol (GPI) is a complex glycolipid that serves as a membrane anchor for many cell surface proteins and is ubiquitous in eukaryotes. As described herein, the C-terminus of a GPI-anchored protein is linked through a phosphoethanolamine bridge to the GPI anchor domain. The GPI anchor domain comprises a highly conserved core glycan structure comprising, mannose (α1-2) mannose (α1-6) mannose (α1-4) glucosamine (α1-6) myo-inositol (Paulick & Bertozzi, “The Glycosylphosphatidylinositol Anchor: A Complex Membrane-Anchoring Structure for Proteins,” Biochemistry 47(27):6991-7000 (2008), which is hereby incorporated by reference in its entirety). A phospholipid tail attaches the GPI anchor to the cell membrane. The core glycan can be modified with various side chains including, e.g., a phosphoethanolamine group, mannose, galactose, sialic acid, or other sugars.

As used herein, the term “glycosylphosphatidylinositol attachment sequence” or “GPI attachment sequence” refers to an amino acid sequence that signals the covalent modification of a polypeptide sequence with a GPI anchor. In any embodiment, the GPI attachment sequence comprises a stretch of hydrophobic amino acids which is post-translationally cleaved and replaced, via a transamidation reaction, with a GPI anchor (see, e.g., Kinoshita, T., “Glycosylphosphatidylinositol (GPI) Anchors: Biochemistry and Cell Biology: Introduction to a Thematic Review Series,” J. Lipid Res. 57(1):4-5 (2016), which is hereby incorporated by reference in its entirety).

The recombinant nucleic acid construct encoding a kallikrein-2 fusion protein as described herein comprises a second nucleotide sequence encoding a GPI attachment sequence, where the nucleotide sequence encoding the GPI attachment sequence is positioned 3′ to the kallikrein-2 encoding nucleotide sequence. Suitable GPI attachment sequences include, without limitation, attachment sequences found in known GPI anchored proteins. For example, the GPI attachment sequence can be the GPI attachment sequence of an alkaline phosphatase, the GPI attachment sequence of a 5′-nucleotidase, the GPI attachment sequence of an acetylcholinesterase, the GPI attachment sequence of a dipeptidase, the GPI attachment sequence of a LFA-3 (CD58), the GPI attachment sequence of a neural cell adhesion molecule (NCAM), the GPI attachment sequence of a decay accelerating factor (DAF; CD55), the GPI attachment sequence of a CD59, the GPI attachment sequence of a Thy-1 (CD90), the GPI attachment sequence of a CD14, the GPI attachment sequence of a carcinoembryonic antigen (CEA), the GPI attachment sequence of a CD16b, and the GPI attachment sequence of a folate-binding protein (Paulick et al, “The Glycosylphosphatidylinositol Anchor: A Complex Membrane-Anchoring Structure for Proteins,” Biochemistry 47(27):6991-7000 (2008), which is hereby incorporated by reference in its entirety). Table 1 provides various exemplary GPI attachment sequences that can be encoded by the second nucleotide sequence of the recombinant construct as described herein.

TABLE 1
Exemplary GPI Attachment Sequences

| GPI Anchor Domain | | SEQ |
|---|---|---|
| Protein* | Attachment Sequence** | ID NO: |
| Human placental alkaline | TACDLAPPAGTTDAAHPGRSVVPALLPLLAGT | 9 |
| phosphatase (PLAP) | LLLLETATAP | |
| Human placental alkaline | TTDAAHPGRSVVPALLPLLAGTLLLLETATAP | 5 |
| phosphatase (PLAP) | | |
| Human decay accelerating | HETTPNKGSGTTSGTTRLLSGHTCFTLTGLLGT | 10 |
| factor (DAF; CD55) | LVTMGLLT | |
| Human ephrin A4 isoform | PGESGTSGWRGGDTPSPLCLLLLLLLLILRLLRI | 11 |
| (EFNA4) | L | |
| Human ephrin A5 isoform | ESAEPSRGENAAQTPRIPSRLLAILLFLLAMLLT | 12 |
| (EFNA5) | L | |
| Human folate receptor 1 | YAAAMSGAGPWAAWPFLLSLALMLLWLLS | 13 |
| (FOLR1) | | |
| Human ephrin A1 isoform | PEVRVLHSIGHSAAPRLFPLAWTVLLLPLLLLQ | 14 |
| (EFNA1) | TP | |
| Human limbic system | SVRGINGSISLAVPLWLLAASLLCLLSKC | 15 |
| associated membrane | | |
| protein (LSAMP) | | |
| Human reticulon 4 | DSEGSGALPSLTCSLTPLGLALVLWTVLGPC | 16 |
| receptor (RTN4R) | | |
| Human contactin 1 | VSQVKISGAPTLSPSLLGLLLPAFGILVYLEF | 17 |
| (CNTN1) | | |
| Human ephrin A3 isoform | QVPKLEKSISGTSPKREHLPLAVGIAFFLMTFLA | 18 |
| (EFNA3) | S | |
| Human ephrin A2 isoform | EAPEPIFTSNNSCSSPGGCRLFLSTIPVLWTLLGS | 19 |
| (EFNA2) | | |
| CD24 | TNATTKAAGGALQSTASLFVVSLSLLHLYS | 20 |

*see, e.g., Varki A, Cummings RD, Esko JD, et al., editors. Essentials of Glycobiology [Internet], 3rd edition. Cold Spring Harbor (NY): ColdSpring Harbor Laboratory Press; 2015-2017. doi: 10.1101/glycobiology.3e.012 and Gahan et al., “Efficient Glycosylphosphatidylinositol (GPI) Modification of Membrane Proteins Requires a C-Terminal Anchoring Signal of Marginal Hydrophobicity,” J. Biol. Chem. 287(20): 16399-16409 (2012), which are hereby incorporated by reference in their entirety; **bold amino acid is the site of attachment of the GPI (sequence to the right of the space is cleaved from the protein upon anchor addition).

In any embodiment, the second nucleotide sequence of the recombinant construct encodes a GPI attachment sequence comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to any one of the amino acid sequences of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and SEQ ID NO: 20.

Other known human GPI anchor domain proteins from which the GPI attachment sequence can be derived from include, without limitation, melanotransferrin, CD109, cadherin 13 isoform 1 preprotein, reticulon 4 receptor-like 1 precursor, carbonic anhydrase 4 preprotein, neurotrimin isoform 1 precursor, mesothelin isoform 2 preprotein, CD48 antigen isoform 1 precursor, sperm acrosome membrane-associated protein 4 precursor, human reversion-inducing cysteine-rich protein with Kazal motifs isoform 1 precursor, carcino-embryonic antigen-related cell adhesion molecule 8 precursor, UL16-binding protein 2 preproprotein, lymphocyte function-associated antigen 3 isoform, Human decoy receptor, carboxy-peptidase M precursor, ecto-ADP-ribosyl-transferase 3 isoform a precursor, GDNF family receptor alpha-4 isoform b precursor, GDNF family receptor alpha-3 preproprotein, brevican core protein isoform 1 precursor, semaphorin-7A isoform 1 preproprotein, CD177 antigen precursor, oligodendrocyte-myelin glycoprotein precursor, CD160 antigen precursor, and intelectin-1 precursor (see, e.g., Pierleoni et al., “R-PredGPI: A GPI Anchor Predictor,” BMC Bioinformatics 9:392 (2008), which is hereby incorporated by reference in its entirety). Accordingly, the second nucleotide sequence of the recombinant construct as described herein can encode a GPI attachment sequence derived from any one of the aforementioned GPI anchor domain protiens.

In any embodiment, the second nucleotide sequence encoding the GPI attachment sequence encodes a GPI attachment sequence derived from alkaline phosphatase. In any embodiment, the second nucleotide sequence encoding the GPI attachment sequence encodes a GPI attachment sequence derived from a human alkaline phosphatase, e.g., a placental alkaline phosphatase, a germ cell alkaline phosphatase, an intestinal-type alkaline phosphatase, or a tissue non-specific alkaline phosphatase.

In any embodiment, the second nucleotide sequence of the recombinant construct encodes the human placental alkaline phosphatase GPI attachment sequence comprising an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 5 or a fragment thereof.

(SEQ ID NO: 5)
TTDAAHPGRSVVPALLPLLAGTLLLLETATAP.

In any embodiment, the second nucleotide sequence of the recombinant construct encodes the human placental alkaline phosphatase GPI attachment sequence of SEQ ID NO: 5 or a fragment thereof.

In any embodiment, the nucleotide sequence encoding the GPI attachment sequence is derived from human placental alkaline phosphatase. For example, the GPI attachment sequence may be derived from human placental alkaline phosphatase (see e.g., GenBank Accession Nos. AAA51706.1, AAA51708.1, or AAA51709.1). In any embodiment, the nucleotide sequence encoding the human placental alkaline phosphatase GPI attachment sequence comprises a nucleotide sequence having 80%, 81%, 82%, 83%, 84%, 85%, 86%, 87%, 88%, 89%, 90%, 91%, 92%, 93%, 94%, 95%, 96%, 97%, 98%, 99% or more sequence identity to the nucleotide sequence of SEQ ID NO: 2.

(SEQ ID NO: 2)
ACCACTGATGCTGCCCATCCTGGAAGGTCTGTGGTGCCTGCCTTGCTGC
CTCTGCTGGCTGGCACTCTGCTGCTGCTGGAGACTGCCACTGCTCCC

In any embodiment, the first and second nucleotide sequences of the construct encode a kallikrein-2 fusion protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 6, as follows:

MWDLVLSIALSVGCTGAVPLIQSRIVGGWECEKHSQPWQVAVYSHGWAH
CGGVLVHPQWVLTAAHCLKKNSQVWLGRHNLFEPEDTGQRVPVSHSFPH
PLYNMSLLKHQSLRPDEDSSHDLMLLRLSEPAKITDVVKVLGLPTQEPA
LGTTCYASGWGSIEPEEFLRPRSLQCVSLHLLSNDMCARAYSEKVTEFM
LCAGLWTGGKDTCGGDSGGPLVCNGVLQGITSWGPEPCALPEKPAVYTK
VVHYRKWIKDTIAANPEFTTDAAHPGRSVVPALLPLLAGTLLLLETATA
P (signal sequence of KLK2 shown in double under-
line; PLAP GPI attachment sequence shown in bold;
cleavage site shown in bold underline).

In any embodiment, the first and second nucleotide sequences of the construct encode a kallikrein-2 fusion protein comprising an amino acid sequence having at least 90% sequence identity to the amino acid sequence of SEQ ID NO: 6. In any embodiment, the the first and second nucleotide sequences of the construct encode the amino acid sequence of SEQ ID NO: 6.

In any embodiment, the first and second nucleotide sequences of the recombinant nucleic acid construct comprises a nucleotide sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 3, as follows:

ATGTGGGACCTGGTTCTCTCCATCGCCTTGTCTGTGGGGTGCACTGGTG
CCGTGCCCCTCATCCAGTCTCGGATCGTGGGGGGCTGGGAGTGCGAGAA
GCACAGCCAGCCTTGGCAAGTGGCAGTGTACTCCCACGGTTGGGCGCAC
TGCGGTGGCGTGCTGGTGCACCCACAATGGGTGCTCACCGCGGCCCACT
GTCTGAAGAAGAATTCACAAGTCTGGCTGGGACGCCATAACCTGTTCGA
ACCTGAAGATACTGGGCAGCGCGTGCCGGTGTCCCATTCCTTCCCTCAC
CCATTGTACAACATGTCGCTGCTGAAGCACCAGTCTTTGAGGCCTGATG
AGGACAGCTCCCATGACCTCATGCTGCTTAGACTCTCGGAACCCGCAAA
GATTACCGACGTCGTGAAAGTGCTTGGACTGCCGACGCAGGAACCCGCC
TTGGGGACTACCTGTTATGCTTCCGGCTGGGGATCCATCGAGCCCGAAG
AATTCCTGCGGCCGCGCAGCCTGCAGTGCGTGTCCCTCCATCTGCTGTC
AAACGATATGTGCGCCAGAGCCTACTCCGAAAAGGTCACCGAGTTTATG
CTGTGCGCCGGACTGTGGACCGGGGGAAAGGACACTTGCGGCGGAGACA
GCGGCGGCCCCCTGGTCTGCAACGGCGTGCTGCAGGGAATTACCTCGTG
GGGTCCAGAGCCGTGTGCGCTGCCTGAAAAGCCCGCCGTGTACACTAAG
GTCGTGCACTACCGGAAGTGGATCAAGGACACCATCGCCGCGAACCCGG
AATTCACCACTGATGCTGCCCATCCTGGAAGGTCTGTGGTGCCTGCCTT
GCTGCCTCTGCTGGCTGGCACTCTGCTGCTGCTGGAGACTGCCACTGCT
CCCTAATGA

(Sequence encoding KLK2 signal sequence shown in double underline; PLAP GPI attachment sequence coding sequence shown in bold; stop codons shown in italic). In any embodiment, the recombinant nucleic acid construct comprises a nucleotide sequence having at least 90% sequence identity to the nucleotide sequence of SEQ ID NO:3. In any embodiment, the recombinant nucleic acid construct comprises the nucleotide sequence of SEQ ID NO: 3.

The recombinant nucleic acid construct of the disclosure is a nucleic acid molecule containing a combination of two or more genetic elements not naturally occurring together. Each recombinant nucleic acid construct may comprise a non-naturally occurring nucleotide sequence that can be in the form of linear DNA, circular DNA, i.e., placed within a vector (e.g., a bacterial vector, a viral vector, plasmid vector), or integrated into a genome. Thus, the nucleic acid constructs of the present disclosure may further comprise a promoter nucleotide sequence positioned 5′ to the KLK2 encoding nucleotide sequence. A promoter is a DNA sequence which contains the binding site for RNA polymerase and initiates transcription of a downstream nucleic acid sequence. Thus, in any embodiment, the nucleic acid constructs described herein comprises a promoter nucleotide sequence.

The promoter may be a constitutively active promoter (i.e., a promoter that is constitutively in an active or “on” state), an inducible promoter (i.e., a promoter whose state, active or inactive state, is controlled by an external stimulus, e.g., the presence of a particular temperature, compound, or protein.), a spatially restricted promoter (i.e., transcriptional control element, enhancer, etc.) (e.g., tissue specific promoter, cell type specific promoter, etc.), or a temporally restricted promoter (i.e., the promoter is in the “on” state or “off” state during specific stages of a biological process).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., RNA Polymerase I, RNA Polymerase II, RNA Polymerase III). The promoter may be a viral promoter. Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., “U6 Promoter-Driven siRNAs with Four Uridine 3′ Overhangs Efficiently Suppress Targeted Gene Expression in Mammalian Cells,” Nat. Biotechnol. 20:497-500 (2002), which is hereby incorporated by reference in its entirety), an enhanced U6 promoter (e.g., Xia et al., “An Enhanced U6 Promoter for Synthesis of Short Hairpin RNA,” Nucleic Acids Res. 31(17):e100 (2003), which is hereby incorporated by reference in its entirety), a human H1 promoter (“H1”), and the like. In any embodiment, the promoter is a phage promoter, e.g., a T7 promoter that has been engineered to be expressed in a mammalian cell.

In any embodiment, the promoter is a eukaryotic RNA polymerase promoter or a derivative thereof. Exemplary RNA polymerase II promoters include, without limitation, cytomegalovirus (“CMV”), phosphoglycerate kinase-1 (“PGK-1”), and elongation factor 1α (“EF1α”) promoters. In yet another embodiment, the promoter is a eukaryotic RNA polymerase III promoter selected from the group consisting of U6, H1, 56, 7SK, and derivatives thereof.

The RNA Polymerase promoter may be mammalian. Suitable mammalian promoters are well known in the art and include, without limitation, human, murine, bovine, canine, feline, ovine, porcine, ursine, and simian promoters.

In any embodiment, the promoter nucleotide sequence is an elongation factor 1 alpha (EF1α) promoter nucleotide sequence. An exemplary EF1α promoter nucleotide sequence is provided as SEQ ID NO: 21 below. Alternatively suitable promoter nucleotide sequences are provided in Table 2 below.

TABLE 2
Exemplary Promoter Nucleotide Sequences

| | | SEQ |
|---|---|---|
| Promoter | Sequence | ID NO: |
| EF1a | GGCTCCGGTGCCCGTCAGTGGGCAGAGCGCACATCGCCCACAGTCCC | 21 |
| | CGAGAAGTTGGGGGGAGGGGTCGGCAATTGAACCGGTGCCTAGAGAA | |
| | GGTGGCGCGGGGTAAACTGGGAAAGTGATGTCGTGTACTGGCTCCGC | |
| | CTTTTTCCCGAGGGTGGGGGAGAACCGTATATAAGTGCAGTAGTCGC | |
| | CGTGAACGTTCTTTTTCGCAACGGGTTTGCCGCCAGAACACAGGTAA | |
| | GTGCCGTGTGTGGTTCCCGCGGGCCTGGCCTCTTTACGGGTTATGGC | |
| | CCTTGCGTGCCTTGAATTACTTCCACCTGGCTGCAGTACGTGATTCT | |
| | TGATCCCGAGCTTCGGGTTGGAAGTGGGTGGGAGAGTTCGAGGCCTT | |
| | GCGCTTAAGGAGCCCCTTCGCCTCGTGCTTGAGTTGAGGCCTGGCCT | |
| | GGGCGCTGGGGCCGCCGCGTGCGAATCTGGTGGCACCTTCGCGCCTG | |
| | TCTCGCTGCTTTCGATAAGTCTCTAGCCATTTAAAATTTTTGATGAC | |
| | CTGCTGCGACGCTTTTTTTCTGGCAAGATAGTCTTGTAAATGCGGGC | |
| | CAAGATCTGCACACTGGTATTTCGGTTTTTGGGGCCGCGGGCGGCGA | |
| | CGGGGCCCGTGCGTCCCAGCGCACATGTTCGGCGAGGCGGGGCCTGC | |
| | GAGCGCGGCCACCGAGAATCGGACGGGGGTAGTCTCAAGCTGGCCGG | |
| | CCTGCTCTGGTGCCTGGTCTCGCGCCGCCGTGTATCGCCCCGCCCTG | |
| | GGCGGCAAGGCTGGCCCGGTCGGCACCAGTTGCGTGAGCGGAAAGAT | |
| | GGCCGCTTCCCGGCCCTGCTGCAGGGAGCTCAAAATGGAGGACGCGG | |
| | CGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGG | |
| | CGCCGTCCAGGCACCTCGATTAGTTCTCGAGCTTTTGGAGTACGTCG | |
| | TCTTTAGGTTGGGGGGAGGGGTTTTATGCGATGGAGTTTCCCCACAC | |
| | TGAGTGGGTGGAGACTGAAGTTAGGCCAGCTTGGCACTTGATGTAAT | |
| | TCTCCTTGGAATTTGCCCTTTTTGAGTTTGGATCTTGGTTCATTCTC | |
| | AAGCCTCAGACAGTGGTTCAAAGTTTTTTTCTTCCATTTCAGGTGTC | |
| | GTGA | |
| CMV | TAGTTATTAATAGTAATCAATTACGGGGTCATTAGTTCATAGCCCAT | 22 |
| | ATATGGAGTTCCGCGTTACATAACTTACGGTAAATGGCCCGCCTGGC | |
| | TGACCGCCCAACGACCCCCGCCCATTGACGTCAATAATGACGTATGT | |
| | TCCCATAGTAACGCCAATAGGGACTTTCCATTGACGTCAATGGGTGG | |
| | AGTATTTACGGTAAACTGCCCACTTGGCAGTACATCAAGTGTATCAT | |
| | ATGCCAAGTACGCCCCCTATTGACGTCAATGACGGTAAATGGCCCGC | |
| | CTGGCATTATGCCCAGTACATGACCTTATGGGACTTTCCTACTTGGC | |
| | AGTACATCTACGTATTAGTCATCGCTATTACCATGGTGATGCGGTTT | |
| | TGGCAGTACATCAATGGGCGTGGATAGCGGTTTGACTCACGGGGATT | |
| | TCCAAGTCTCCACCCCATTGACGTCAATGGGAGTTTGTTTTGGCACC | |
| | AAAATCAACGGGACTTTCCAAAATGTCGTAACAACTCCGCCCCATTG | |
| | ACGCAAATGGGCGGTAGGCGTGTACGGTGGGAGGTCTATATAAGCAG | |
| | AGCTGGTTTAGTGAACCGTCAGATC | |
| CAGG | ACTAGTTATTAATAGTAATCAATTACGGGGTCATTAGTTCATAGCCC | 23 |
| | ATATATGGAGTTCCGCGTTACATAACTTACGGTAAATGGCCCGCCTG | |
| | GCTGACCGCCCAACGACCCCCGCCCATTGACGTCAATAATGACGTAT | |
| | GTTCCCATAGTAACGCCAATAGGGACTTTCCATTGACGTCAATGGGT | |
| | GGAGTATTTACGGTAAACTGCCCACTTGGCAGTACATCAAGTGTATC | |
| | ATATGCCAAGTACGCCCCCTATTGACGTCAATGACGGTAAATGGCCC | |
| | GCCTGGCATTATGCCCAGTACATGACCTTATGGGACTTTCCTACTTG | |
| | GCAGTACATCTACGTATTAGTCATCGCTATTACCATGGTCGAGGTGA | |
| | GCCCCACGTTCTGCTTCACTCTCCCCATCTCCCCCCCCTCCCCACCC | |
| | CCAATTTTGTATTTATTTATTTTTTAATTATTTTGTGCAGCGATGGG | |
| | GGCGGGGGGGGGGGGGGGGCGCGCGCCAGGCGGGGCGGGGCGGGGCG | |
| | AGGGGCGGGGCGGGGCGAGGCGGAGAGGTGCGGCGGCAGCCAATCAG | |
| | AGCGGCGCGCTCCGAAAGTTTCCTTTTATGGCGAGGCGGCGGCGGCG | |
| | GCGGCCCTATAAAAAGCGAAGCGCGCGGCGGGCGGGGAGTCGCTGCG | |
| | ACGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACG | |
| | GCTTGTTTCTTTTCTGTGGCTGCGTGAAAGCCTTGAGGGGCTCCGGG | |
| | AGGGCCCTTTGTGCGGGGGGAGCGGCTCGGGGGGTGCGTGCGTGTGT | |
| | GTGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGGGC | |
| | TGCGAGGGGAACAAAGGCTGCGTGCGGGGTGTGTGCGTGGGGGGGTG | |
| | AGCAGGGGGTGTGGGCGCGTCGGTCGGGCTGCAACCCCCCCTGCACC | |
| | CCCCTCCCCGAGTTGCTGAGCACGGCCCGGCTTCGGGTGCGGGGCTC | |
| | CGTACGGGGCGTGGCGCGGGGCTCGCCGTGCCGGGCGGGGGGTGGCG | |
| | GCAGGTGGGGGTGCCGGGCGGGGCGGGGCCGCCTCGGGCCGGGGAGG | |
| | GCTCGGGGGAGGGGCGCGGCGGCCCCCGGAGCGCCGGCGGCTGTCGA | |
| | GGCGCGGCGAGCCGCAGCCATTGCCTTTTATGGTAATCGTGCGAGAG | |
| | GGCGCAGGGACTTCCTTTGTCCCAAATCTGTGCGGAGCCGAAATCTG | |
| | GGAGGCGCCGCCGCACCCCCTCTAGCGGGCGCGGGGCGAAGCGGTGC | |
| | GGCGCCGGCAGGAAGGAAATGGGCGGGGAGGGCCTTCGTGCGTCGCC | |
| | GCGCCGCCGTCCCCTTCTCCCTCTCCAGCCTCGGGGCTGTCCGCGGG | |
| | GGGACGGCTGCCTTCGGGGGGGACGGGGCAGGGCGGGGTTCGGCTTC | |
| | TGGCGTGTGACCGGCGGCTCTAGAGCCTCTGCTAACCATGTTCATGC | |
| | CTTCTTCTTTTTCCTACAGCTCCTGGGCAACGTGCTGGTTATTGTGC | |
| | TGTCTCATCATTTTGGCAAAGAATTC | |
| PGK1 | TTCTACCGGGTAGGGGAGGCGCTTTTCCCAAGGCAGTCTGGAGCATG | 24 |
| | CGCTTTAGCAGCCCCGCTGGGCACTTGGCGCTACACAAGTGGCCTCT | |
| | GGCCTCGCACACATTCCACATCCACCGGTAGGCGCCAACCGGCTCCG | |
| | TTCTTTGGTGGCCCCTTCGCGCCACCTTCTACTCCTCCCCTAGTCAG | |
| | GAAGTTCCCCCCCGCCCCGCAGCTCGCGTCGTGCAGGACGTGACAAA | |
| | TGGAAGTAGCACGTCTCACTAGTCTCGTGCAGATGGACAGCACCGCT | |
| | GAGCAATGGAAGCGGGTAGGCCTTTGGGGCAGCGGCCAATAGCAGCT | |
| | TTGCTCCTTCGCTTTCTGGGCTCAGAGGCTGGGAAGGGGTGGGTCCG | |
| | GGGGCGGGCTCAGGGGCGGGCTCAGGGGCGGGGCGGGCGCCCGAAGG | |
| | TCCTCCGGAGGCCCGGCATTCTGCACGCTTCAAAAGCGCACGTCTGC | |
| | CGCGCTGTTCTCCTCTTCCTCATCTCCGGGCCTTTCGACCT | |
| SV40 | CTGTGGAATGTGTGTCAGTTAGGGTGTGGAAAGTCCCCAGGCTCCCC | 25 |
| | AGCAGGCAGAAGTATGCAAAGCATGCATCTCAATTAGTCAGCAACCA | |
| | GGTGTGGAAAGTCCCCAGGCTCCCCAGCAGGCAGAAGTATGCAAAGC | |
| | ATGCATCTCAATTAGTCAGCAACCATAGTCCCGCCCCTAACTCCGCC | |
| | CATCCCGCCCCTAACTCCGCCCAGTTCCGCCCATTCTCCGCCCCATG | |
| | GCTGACTAATTTTTTTTATTTATGCAGAGGCCGAGGCCGCCTCTGCC | |
| | TCTGAGCTATTCCAGAAGTAGTGAGGAGGCTTTTTTGGAGGCCTAGG | |
| | CTTTTGCAAAAAGCT | |
| UBC | GGTGCAGCGGCCTCCGCGCCGGGTTTTGGCGCCTCCCGCGGGCGCCC | 26 |
| | CCCTCCTCACGGCGAGCGCTGCCACGTCAGACGAAGGGCGCAGGAGC | |
| | GTTCCTGATCCTTCCGCCCGGACGCTCAGGACAGCGGCCCGCTGCTC | |
| | ATAAGACTCGGCCTTAGAACCCCAGTATCAGCAGAAGGACATTTTAG | |
| | GACGGGACTTGGGTGACTCTAGGGCACTGGTTTTCTTTCCAGAGAGC | |
| | GGAACAGGCGAGGAAAAGTAGTCCCTTCTCGGCGATTCTGCGGAGGG | |
| | ATCTCCGTGGGGCGGTGAACGCCGATGATTATATAAGGACGCGCCGG | |
| | GTGTGGCACAGCTAGTTCCGTCGCAGCCGGGATTTGGGTCGCGGTTC | |
| | TTGTTTGTGGATCGCTGTGATCGTCACTTGGTGAGTTGCGGGCTGCT | |
| | GGGCTGGCCGGGGCTTTCGTGGCCGCCGGGCCGCTCGGTGGGACGGA | |
| | AGCGTGTGGAGAGACCGCCAAGGGCTGTAGTCTGGGTCCGCGAGCAA | |
| | GGTTGCCCTGAACTGGGGGTTGGGGGGAGCGCACAAAATGGCGGCTG | |
| | TTCCCGAGTCTTGAATGGAAGACGCTTGTAAGGCGGGCTGTGAGGTC | |
| | GTTGAAACAAGGTGGGGGGCATGGTGGGCGGCAAGAACCCAAGGTCT | |
| | TGAGGCCTTCGCTAATGCGGGAAAGCTCTTATTCGGGTGAGATGGGC | |
| | TGGGGCACCATCTGGGGACCCTGACGTGAAGTTTGTCACTGACTGGA | |
| | GAACTCGGGTTTGTCGTCTGGTTGCGGGGGCGGCAGTTATGCGGTGC | |
| | CGTTGGGCAGTGCACCCGTACCTTTGGGAGCGCGCGCCTCGTCGTGT | |
| | CGTGACGTCACCCGTTCTGTTGGCTTATAATGCAGGGTGGGGCCACC | |
| | TGCCGGTAGGTGTGCGGTAGGCTTTTCTCCGTCGCAGGACGCAGGGT | |
| | TCGGGCCTAGGGTAGGCTCTCCTGAATCGACAGGCGCCGGACCTCTG | |
| | GTGAGGGGAGGGATAAGTGAGGCGTCAGTTTCTTTGGTCGGTTTTAT | |
| | GTACCTATCTTCTTAAGTAGCTGAAGCTCCGGTTTTGAACTATGCGC | |
| | TCGGGGTTGGCGAGTGTGTTTTGTGAAGTTTTTTAGGCACCTTTTGA | |
| | AATGTAATCATTTGGGTCAATATGTAATTTTCAGTGTTAGACTAGTA | |
| | AA | |

Some embodiments of the present disclosure relate to a vector comprising the recombinant nucleic acid construct as described herein (i.e., a recombinant nucleic acid construct encoding a kallikrein-2 fusion protein, said construct comprising: a nucleotide sequence encoding kallikrein-2 (KLK2) and a nucleotide sequence encoding a glycosylphophatidylinositol (GPI) attachment sequence, where said GPI attachment sequence encoding nucleotide sequence is positioned 3′ to the KLK2 encoding nucleotide sequence). As used herein, the term vector means any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. Thus, in some embodiments, the recombinant nucleic acid construct may be inserted into an expression system or vector in proper sense (5′ to 3′) orientation and correct reading frame. The vector may contain the necessary elements for the transcription and/or translation of the kallikrein-2 fusion protein as disclosed herein.

In one embodiment, the vector is a plasmid. Numerous vectors suitable for containing the recombinant nucleic acid construct disclosed herein are known to those of skill in the art, and many are commercially available. The following vectors are provided by way of example; for eukaryotic cells: pcDNA3.1(+), Tornado (Litke & Jaffrey, “Highly Efficient Expression of Circular RNA Aptamers in Cells Using Autocatalytic Transcripts,” Nat. Biotechnol. 37(6):667-675(2019), which is hereby incorporated by reference in its entirety), pXT1, pSG5 (Stratagene), pSVK3, pBPV, pMSG, and pSVLSV40 (Pharmacia). However, any other vector may be used so long as it is compatible with the cell.

In another embodiment, the vector is a viral vector. The viral vector may be selected from any vector suitable for introduction of the recombinant nucleic acid construct described herein into a cell by any means to facilitate the expression of the recombinant nucleic acid construct. Suitable viral vectors include, but are not limited to, viral vectors based on vaccinia virus; poliovirus; adenovirus (see, e.g., PCT Patent Application Publication Nos. WO 94/12649 to Gregory et al., WO 93/03769 to Crystal et al., WO 93/19191 to Haddada et al., WO 94/28938 to Wilson et al., WO 95/11984 to Gregory, and WO 95/00655 to Graham, which are hereby incorporated by reference in their entirety); adeno-associated virus (see, e.g., Flannery et al., “Efficient Photoreceptor-Targeted Gene Expression In Vivo by Recombinant Adeno-Associated Virus,” PNAS 94:6916-6921 (1997); Bennett et al., “Real-Time, Noninvasive In Vivo Assessment of Adeno-Associated Virus-Mediated Retinal Transduction,” Invest. Opthalmol. Vis. Sci. 38:2857-2863 (1997); Jomary et al., “Nonviral Ocular Gene Transfer,” Gene Ther. 4:683-690 (1997); Rolling et al., “Evaluation of Adeno-Associated Virus-Mediated Gene Transfer into the Rat Retina by Clinical Fluorescence Photography,” Hum. Gene. Ther. 10:641-648 (1999); Ali et al., “Gene Transfer Into the Mouse Retina Mediated by an Adeno-Associated Viral Vector,” Hum. Mol. Genet. 5:591-594 (1996); Samulski et al., “Helper-Free Stocks of Recombinant Adeno-Associated Viruses: Normal Integration Does not Require Viral Gene Expression,” J. Vir. 63:3822-3828 (1989); Mendelson et al., “Expression and Rescue of a Nonselected Marker from an Integrated AAV Vector,” Virol. 166:154-165 (1988); and Flotte et al., “Stable In Vivo Expression of the Cystic Fibrosis Transmembrane Conductance Regulator With an Adeno-Associated Virus Vector,” PNAS 90:10613-10617 (1993), which are hereby incorporated by reference in their entirety); SV40; herpes simplex virus; human immunodeficiency virus (see, e.g., Miyoshi et al., “Stable and Efficient Gene Transfer into the Retina Using an HIV-Based Lentiviral Vector,” PNAS 94:10319-10323 (1997), which is hereby incorporated by reference in its entirety); a retroviral vector, e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus and the like. Thus, in some embodiments, the viral vector is selected from the group consisting of an adenoviral vector, an adeno-associated viral vector, a lentiviral vector, a vaccina vector, a retroviral vector, and a herpes simplex viral vector.

An exemplary viral vector comprising the KLK2-GPI recombinant construct has the sequence of SEQ ID NO: 7, as follows:

ACGCGTGTAGTCTTATGCAATACTCTTGTAGTCTTGCAACATGGTAACGATGAGTTAGCAACAT
GCCTTACAAGGAGAGAAAAAGCACCGTGCATGCCGATTGGTGGAAGTAAGGTGGTACGATCGTG
CCTTATTAGGAAGGCAACAGACGGGTCTGACATGGATTGGACGAACCACTGAATTGCCGCATTG
CAGAGATATTGTATTTAAGTGCCTAGCTCGATACATAAACGGGTCTCTCTGGTTAGACCAGATC
TGAGCCTGGGAGCTCTCTGGCTAACTAGGGAACCCACTGCTTAAGCCTCAATAAAGCTTGCCTT
GAGTGCTTCAAGTAGTGTGTGCCCGTCTGTTGTGTGACTCTGGTAACTAGAGATCCCTCAGACC
CTTTTAGTCAGTGTGGAAAATCTCTAGCAGTGGCGCCCGAACAGGGACTTGAAAGCGAAAGGGA
AACCAGAGGAGCTCTCTCGACGCAGGACTCGGCTTGCTGAAGCGCGCACGGCAAGAGGCGAGGG
GCGGCGACTGGTGAGTACGCCAAAATTTGACTAGCGGAGGCTAGAGGGAGAGAGATGGGTGCGA
GAGCGTCAGTATTAAGCGGGGGAGAATAAGATCGCGATGGGAAAAAATTCGGTTAAGGCCAGGG
GGAAAGAAAAAATATAAATTAAAACATATAGTATGGGCAAGCAGGGAGCTAGAACGATTCGCAG
TTAATCCTGGCCTGTTAGAAACATCAGAAGGCTGTAGACAAATACTGGGACAGCTACAACCATC
CCTTCAGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGCAAAACA
AAAGTAAGACCACCGCACAGCAAGCGGCCACTGATCTTCAGACCTGGAGGAGGAGATATGAGGG
ACAATTGGAGAAGTGAATTATATAAATATAAAGTAGTAAAAATTGAACCATTAGGAGTAGCACC
CACCAAGGCAAAGAGAAGAGTGGTGCAGAGAGAAAAAAGAGCAGTGGGAATAGGAGCTTTGTTC
CTTGGGTTCTTGGGAGCAGCAGGAAGCACTATGGGCGCAGCGTCAATGACGCTGACGGTACAGG
CCAGACAATTATTGTCTGGTATAGTGCAGCAGCAGAACAATTTGCTGAGGGCTATTGAGGCGCA
ACAGCATCTGTTGCAACTCACAGTCTGGGGCATCAAGCAGCTCCAGGCAAGAATCCTGGCTGTG
GAAAGATACCTAAAGGATCAACAGCTCCTGGGGATTTGGGGTTGCTCTGGAAAACTCATTTGCA
CCACTGCTGTGCCTTGGAATGCTAGTTGGAGTAATAAATCTCTGGAACAGATTTGGAATCACAC
GACCTGGATGGAGTGGGACAGAGAAATTAACAATTACACAAGCTTAATACACTCCTTAATTGAA
GAATCGCAAAACCAGCAAGAAAAGAATGAACAAGAATTATTGGAATTAGATAAATGGGCAAGTT
TGTGGAATTGGTTTAACATAACAAATTGGCTGTGGTATATAAAATTATTCATAATGATAGTAGG
AGGCTTGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCGACAGGCCCGAAGGAA
TAGAAGAAGAAGGTGGAGAGAGAGACAGAGACAGATCCATTCGATTAGTGAACGGATCTCGACG
GTATCGGTTAACTTTTAAAAGAAAAGGGGGGATTGGGGGGTACAGTGCAGGGGAAAGAATAGTA
GACATAATAGCAACAGACATACAAACTAAAGAATTACAAAAACAAATTACAAAATTCAAAATTT
TTCGATACTAGTGGATCTGCGATCGCTCCGGTGCCCGTCAGTGGGCAGAGCGCACATCGCCCAC
AGTCCCCGAGAAGTTGGGGGGAGGGGTCGGCAATTGAACGGGTGCCTAGAGAAGGTGGCGCGGG
GTAAACTGGGAAAGTGATGTCGTGTACTGGCTCCGCCTTTTTCCCGAGGGTGGGGGAGAACCGT
ATATAAGTGCAGTAGTCGCCGTGAACGTTCTTTTTCGCAACGGGTTTGCCGCCAGAACACAGCT
GAAGCTTCGAGGGGCTCGCATCTCTCCTTCACGCGCCCGCCGCCCTACCTGAGGCCGCCATCCA
CGCCGGTTGAGTCGCGTTCTGCCGCCTCCCGCCTGTGGTGCCTCCTGAACTGCGTCCGCCGTCT
AGGTAAGTTTAAAGCTCAGGTCGAGACCGGGCCTTTGTCCGGCGCTCCCTTGGAGCCTACCTAG
ACTCAGCCGGCTCTCCACGCTTTGCCTGACCCTGCTTGCTCAACTCTACGTCTTTGTTTCGTTT
TCTGTTCTGCGCCGTTACAGATCCAAGCTGTGACCGGCGCCTACTCTAGAGCCGCCACCATGTG
GGACCTGGTTCTCTCCATCGCCTTGTCTGTGGGGTGCACTGGTGCCGTGCCCCTCATCCAGTCT
CGGATCGTGGGGGGCTGGGAGTGCGAGAAGCACAGCCAGCCTTGGCAAGTGGCAGTGTACTCCC
ACGGTTGGGCGCACTGCGGTGGCGTGCTGGTGCACCCACAATGGGTGCTCACCGCGGCCCACTG
TCTGAAGAAGAATTCACAAGTCTGGCTGGGACGCCATAACCTGTTCGAACCTGAAGATACTGGG
CAGCGCGTGCCGGTGTCCCATTCCTTCCCTCACCCATTGTACAACATGTCGCTGCTGAAGCACC
AGTCTTTGAGGCCTGATGAGGACAGCTCCCATGACCTCATGCTGCTTAGACTCTCGGAACCCGC
AAAGATTACCGACGTCGTGAAAGTGCTTGGACTGCCGACGCAGGAACCCGCCTTGGGGACTACC
TGTTATGCTTCCGGCTGGGGATCCATCGAGCCCGAAGAATTCCTGCGGCCGCGCAGCCTGCAGT
GCGTGTCCCTCCATCTGCTGTCAAACGATATGTGCGCCAGAGCCTACTCCGAAAAGGTCACCGA
GTTTATGCTGTGCGCCGGACTGTGGACCGGGGGAAAGGACACTTGCGGCGGAGACAGCGGCGGC
CCCCTGGTCTGCAACGGCGTGCTGCAGGGAATTACCTCGTGGGGTCCAGAGCCGTGTGCGCTGC
CTGAAAAGCCCGCCGTGTACACTAAGGTCGTGCACTACCGGAAGTGGATCAAGGACACCATCGC
CGCGAACCCGGAATTCACCACTGATGCTGCCCATCCTGGAAGGTCTGTGGTGCCTGCCTTGCTG
CCTCTGCTGGCTGGCACTCTGCTGCTGCTGGAGACTGCCACTGCTCCCTAATGAGGATCCGCGG
CCGCGCCCCTCTCCCTCCCCCCCCCCTAACGTTACTGGCCGAAGCCGCTTGGAATAAGGCCGGT
GTGCGTTTGTCTATATGTTATTTTCCACCATATTGCCGTCTTTTGGCAATGTGAGGGCCCGGAA
ACCTGGCCCTGTCTTCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGAAGACAAACAACGTCTG
TAGCGACCCTTTGCAGGCAGCGGAACCCCCCACCTGGCGACAGGTGCCTCTGCGGCCAAAAGCC
ACGTGTATAAGATACACCTGCAAAGGCGGCACAACCCCAGTGCCACGTTGTGAGTTGGATAGTT
GTGGAAAGAGTCAAATGGCTCTCCTCAAGCGTATTCAACAAGGGGCTGAAGGATGCCCAGAAGG
TACCCCATTGTATGGGATCTGATCTGGGGCCTCGGTGCACATGCTTTACATGTGTTTAGTCGAG
GTTAAAAAAACGTCTAGGCCCCCCGAACCACGGGGACGTGGTTTTCCTTTGAAAAACACGATGA
TAATATGGCCACAACCATGGCGTCCGGAATGATTGAACAAGATGGATTGCACGCAGGTTCTCCG
GCCGCTTGGGTGGAGAGGCTATTCGGCTATGACTGGGCACAACAGACAATCGGCTGCTCTGATG
CCGCCGTGTTCCGGCTGTCAGCGCAGGGGCGCCCGGTTCTTTTTGTCAAGACCGACCTGTCCGG
TGCCCTGAATGAACTGCAGGACGAGGCAGCGCGGCTATCGTGGCTGGCCGCGACGGGCGTTCCT
TGCGCAGCTGTGCTCGACGTTGTCACTGAAGCGGGAAGGGACTGGCTGCTATTGGGCGAAGTGC
CGGGGCAGGATCTCCTGTCATCTCACCTTGCTCCTGCCGAGAAAGTATCCATCATGGCTGATGC
AATGCGGCGGCTGCATACGCTTGATCCGGCTACCTGCCCATTCGACCACCAAGCGAAACATCGC
ATCGAGCGAGCACGTACTCGGATGGAAGCCGGTCTTGTCGATCAGGATGATCTGGACGAAGAGC
ATCAGGGGCTCGCGCCAGCCGAACTGTTCGCCAGGCTCAAGGCGCGCATGCCCGACGGCGAGGA
TCTCGTCGTGACCCATGGCGATGCCTGCTTGCCGAATATCATGGTGGAAAATGGCCGCTTTTCT
GGATTCATCGACTGTGGCCGGCTGGGTGTGGCGGACCGCTATCAGGACATAGCGTTGGCTACCC
GTGATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGTCGACTCG
ACAATCAACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTG
TCAGGCAACGTGGCGTGGTGTGCACTGTGTTTGCTGACGCAACCCCCACTGGTTGGGGCATTGC
CACCACCTGTCAGCTCCTTTCCGGGACTTTCGCTTTCCCCCTCCCTATTGCCACGGCGGAACTC
ATCGCCGCCTGCCTTGCCCGCTGCTGGACAGGGGCTCGGCTGTTGGGCACTGACAATTCCGTGG
TGTTGTCGGGGAAATCATCGTCCTTTCCTTGGCTGCTCGCCTGTGTTGCCACCTGGATTCTGCG
CGGGACGTCCTTCTGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGTCGGATCTCCCTTT
GGGCCGCCTCCCCGCCTGGTACCTTTAAGACCAATGACTTACAAGGCAGCTGTAGATCTTAGCC
ACTTTTTAAAAGAAAAGGGGGGACTGGAAGGGCTAATTCACTCCCAACGAAGATAAGATCTGCT
TTTTGCTTGTACTGGGTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAGTAGTGTGTGCCCGTC
TGTTGTGTGACTCTGGTAACTAGAGATCCCTCAGACCCTTTTAGTCAGTGTGGAAAATCTCTAG
CAGTAGTAGTTCATGTCATCTTATTATTCAGTATTTATAACTTGCAAAGAAATGAATATCAGAG
AGTGAGAGGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATC
TTATCATGTCTGGCTCTAGCTATCCCGCCCCTAACTCCGCCCATCCCGCCCCTAACTCCGCCCA
GTTCCGCCCATTCTCCGCCCCATGGCTGACTAATTTTTTTTATTTATGCAGAGGCCGAGGCCGC
CTCGGCCTCTGAGCTATTCCAGAAGTAGTGAGGAGGCTTTTTTGGAGGCCTAGACTTTTGCAGA
GACCAAATTCGTAATCATGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTCAAAGGCGGTAA
TACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATACCT
GTCCGCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAG
TGGTGGCCTAACTACGGCTACACTAGAAGGACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGAT
TATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACCGGCTCCAGA
TTTATCAGCAATAAACCAGCCAGCCGGAAGGGCCGAGCGCAGAAGTGGTCCTGCAACTTTATCC
GCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTTGCCCGGCG
TCAATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATATTTGAATG
TATTTAGAAAAATAAACAAATAGGGGTTCCGCGCACATTTCCCCGAAAAGTGCCACCTGACGTC
TAAGAAACCATTATTATCATGACATTAACCTATAAAAATAGGCGTATCACGAGGCCCTTTCGTC
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATATGCGGT
GTGAAATACCGCACAGATGCGTAAGGAGAAAATACCGCATCAGGCGCCATTCGCCATTCAGGCT
GCGCAACTGTTGGGAAGGGCGATCGGTGCGGGCCTCTTCGCTATTACGCCAGCTGGCGAAAGGG
GGATGTGCTGCAAGGCGATTAAGTTGGGTAACGCCAGGGTTTTCCCAGTCACGACGTTGTAAAA
CGACGGCCAGTGCCAAGCTG (pCDH Neo vector encoding huKLK2_GPI;
SEQ ID NO: 7)

Another aspect of the present disclosure relates to a kallikrein-2 fusion protein encoded by a recombinant nucleic acid construct as described herein or a vector comprising the recombinant nucleic acid construct according to the present disclosure.

Thus, in any embodiment, the kallikrein-2 fusion protein according to the present disclosure comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 6, as follows:

MWDLVLSIALSVGCTGAVPLIQSRIVGGWECEKHSQPWQVAVYSHGWAH
CGGVLVHPQWVLTAAHCLKKNSQVWLGRHNLFEPEDTGQRVPVSHSFPH
PLYNMSLLKHQSLRPDEDSSHDLMLLRLSEPAKITDVVKVLGLPTQEPA
LGTTCYASGWGSIEPEEFLRPRSLQCVSLHLLSNDMCARAYSEKVTEFM
LCAGLWTGGKDTCGGDSGGPLVCNGVLQGITSWGPEPCALPEKPAVYTK
VVHYRKWIKDTIAANPEFTTDAAHPGRSVVPALLPLLAGTLLLLETATA
P (signal sequence of KLK2 shown in double under-
line; PLAP GPI attachment sequence shown in bold;
cleavage site shown in bold underline).

In any embodiment, the kallikrein-2 fusion protein disclosed herein comprises an amino acid sequence having at least 90% sequence identity to the amino acid sequence of SEQ ID NO: 6. In any embodiment, the kallikrein-2 fusion protein comprises the amino acid sequence of SEQ ID NO: 6.

As described supra, the glycosylphophatidylinositol (GPI) attachment sequence comprises a stretch of hydrophobic amino acids, which are post-translationally cleaved and replaced, via a transamidation reaction, with a GPI anchor (see, e.g., Kinoshita, T., “Glycosylphosphatidylinositol (GPI) Anchors: Biochemistry and Cell Biology: Introduction to a Thematic Review Series,” J. Lipid Res. 57(1):4-5 (2016), which is hereby incorporated by reference in its entirety). Thus, in any embodiment, the GPI attachment sequence described herein comprise a cleavage site. In accordance with such embodiments, the kallikrein-2 fusion protein according to the present disclosure does not comprise the amino acid residues following the cleavage site. For example, in some embodiments, the kallikrein-2 fusion protein, when expressed in vivo, does not comprise amino acid residues 267-295 of SEQ ID NO:6.

In any embodiment, the kallikrein-fusion protein of the present disclosure protein does not comprise the amino-terminal signal sequence of the kallikrein portion of the fusion protein. Thus, in some embodiments, the kallikrein-fusion protein does not comprise amino acid residues 1-17 of SEQ ID NO:6.

In any embodiment, the kallikrein-2 fusion protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO:7. For example, the kallikrein-2 fusion protein may have an amino acid sequence having at least 90% sequence identity to the amino acid sequence of SEQ ID NO:7. In any embodiment, the kallikrein-2 fusion protein has the amino acid sequence of SEQ ID NO:7.

Another aspect of the present disclosure relates to a preparation of cells, where cells of the preparation are modified to express the recombinant kallikrein-2 fusion construct as described herein. Cells of the preparation are modified to express, on their surface, a recombinant kallikrein-2 fusion protein, where the kallikrein-2 fusion protein includes a kallikrein-2 polypeptide sequence, a portion of a glycosylphophatidylinositol (GPI) attachment sequence linked to the C-terminus of the kallikrein-2 polypeptide sequence, and a GPI anchor domain coupled to the GPI attachment sequence portion.

As described in detail supra, the kallikrein-2 portion of the fusion protein can encompass any mammalian kallikrein-2 polypeptide sequence, e.g., a human, murine, bovine, canine, feline, ovine, porcine, ursine, or simian kallikrein-2 polypeptide sequence. In any embodiment, the kallikrein-2 portion of the fusion protein comprises a human kallikrein-2 protein or polypeptide fragment thereof. For example, the human kallikrein-2 polypeptide sequence may have an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO:4 or amino acid residues 18-263 of SEQ ID NO:4.

The portion of the GPI attachment sequence can be derived from a GPI attachment sequence of a known GPI anchor domain protein. Exemplary GPI anchor domain proteins and GPI attachment sequences are provided supra. In any embodiment, the portion of the GPI attachment sequence is derived from alkaline phosphatase, e.g., human placental alkaline phosphatase.

In any embodiment, the portion of the GPI attachment sequence is a portion of the amino acid sequence of SEQ ID NO:5; or an amino acid sequence having at least 90%, 91%, 92%, 93%, 94%, 95%, 96%, 97%, 98%, 99%, or more sequence identity to the amino acid sequence of SEQ ID NO:5. In any embodiment, the GPI attachment sequence portion of the kallikrein-2 fusion protein as described herein comprises amino acid residues 1-3 of SEQ ID NO:5.

In any embodiment, the preparation of cells are modified to express a recombinant kallikrein-2 fusion protein having the amino acid sequence having at least 90%, 91%, 92%, 93%, 94%, 95%, 96%, 97%, 98%, 99%, or more sequence identity to the amino acid sequence of SEQ ID NO:6 or the amino acid sequence of SEQ ID NO: 7. For example, cells of the preparation may express on their surface a kallikrein-2 fusion protein comprising an amino acid sequence having at least 90% sequence identity to the amino acid sequence of SEQ ID NO: 6 or amino sequence of SEQ ID NO:7.

In other embodiments, the preparation of cells express, on their surface, or are modified with, a recombinant kallikrein-2 fusion protein having the sequence of SEQ ID NO:6 or the amino acid sequence of SEQ ID NO:7.

The expressed kallikrein-2 fusion protein further comprises a GPI anchor domain. The GPI anchor domain is coupled to the GPI attachment sequence via a GPI transamidase reaction that occurs in vivo post-translationally. The attached GPI anchor domain comprises the core glycan structure of ethanolamine-PO-6Manα1-2Manα1-6Manα1-4GlcNα1-6myo-inositol-1-PO-lipid.

As noted supra, the cells of the preparation may express the kallikrein-2 fusion protein from the recombinant nucleic acid construct (e.g., a linear construct) according to the present disclosure or a vector comprising the recombinant nucleic acid construct according to the present disclosure.

The recombinant nucleic acid constructs and/or vectors described herein may be introduced into cells via transformation, particularly transduction, conjugation, lipofection, protoplast fusion, mobilization, particle bombardment, microinjection, transfection, or electroporation. In any embodiment, the cells of the preparation are stably transduced with the nucleic acid construct according to the present disclosure or the vector according to the present disclosure. In any embodiment, the cells of the preparation comprise the recombinant nucleic acid construct stably integrated in their genome.

In any embodiment, the cells of the preparation are mammalian cells. Suitable mammalian cells include, without limitation, rodent cells (i.e., mouse or rat cells), rabbit cells, guinea pig cells, feline cells, canine cells, porcine cells, equine cells, bovine cell, ovine cells, monkey cells, non-human primate, or human cells. In any embodiment, the cells of the preparation are human cells.

Suitable preparations of cells comprising the recombinant nucleic acid constructs or vectors as described herein include primary, immortalized or transformed embryonic cells, fetal cells, or adult cells, at any stage of their lineage, e.g., totipotent, pluripotent, multipotent, or differentiated cells. Additional suitable preparations of cells include cells from a cell line.

In any embodiment, the cells of the preparation are prostate cells, e.g., primary prostate cells, primary prostate cancer cells, prostate cancer cell lines, or non-tumor prostate cell lines.

Suitable exemplary non-tumor prostate cell lines include, without limitation, pRNS-1-1, RWPE-1, BPH1, and PIN cell lines (Cunningham & You, “In Vitro and In Vivo Model Systems Used in Prostate Cancer Research,” J. Biol. Methods 2(1):e17 (2015), which is hereby incorporated by reference in its entirety). RWPE-1 cells were immortalized with human papilloma virus (HPV) 18 with subsequence isolation and propagation over 6-7 weeks and is positive for AR/PSA mRNA/protein and is androgen sensitive. BPH1 cells were isolated from benign prostatic hypertrophy or hyperplasia (BPH) tissues obtained through transurethral resection from a patient undergoing the procedure for urinary obstruction consistent with BPH. BPH1 cells were immortalized with SV40 large T antigen and are AR/PSA negative and WT p53 positive. PIN cells were isolated from a patient with prostatic intraepithelial neoplasia (PIN) and immortalized with HPV 18.

In any embodiment, the prostate cells are hormone naïve prostate cancer (PCa) cells lines. Suitable hormone naïve PCa cell lines include, without limitation, RWPE-2, LNCaP, LAPC-4, LAPC-9, VCaP, MDA PCa 2a/2b, and LuCaP (Cunningham & You, “In Vitro and In Vivo Model Systems Used in Prostate Cancer Research,” J. Biol. Methods 2(1):e17 (2015), which is hereby incorporated by reference in its entirety). LNCaP cells were first isolated from a human metastatic prostate adenocarcinoma found in a lymph node and is androgen responsive with AR and PSA mRNA/protein expression. VCaP cells were first isolated in 2001, as the result of a vertebral metastatic lesion. VCaP cells are positive for androgen sensitivity with wild-type AR mRNA/protein, and express PSA mRNA/protein, prostatic acid phosphatase (PAP), retinoblastoma (Rb), and p53 (with an A248W mutation). MDA PCa 2a/2b cell lines were derived from a single patient with vertebral metastasis during late stage disease, are androgen sensitive and tumorigenic in mice, express AR mRNA/protein, and express PSA mRNA/protein.

In any embodiment, the prostate cancer cell lines are castration resistant cell lines. Suitable castration resistant cell lines include, without limitation, C4-2, C4-2B, 22Rv1, ARCaP (MDA PCa 1), PC3, and DU145 cell lines (Cunningham & You, “In Vitro and In Vivo Model Systems Used in Prostate Cancer Research,” J. Biol. Methods 2(1):e17 (2015), which is hereby incorporated by reference in its entirety). PC3 cells were isolated from a vertebral metastatic prostate tumor, are hormone independent, do not express androgen receptor (AR) or PSA mRNA/protein, and express an aberrant p53 with a C deletion in codon 138 causing a nonsense codon at 169 (causing a loss of heterozygosity). DU145 cells are derived from a brain metastasis, are hormone independent, do not express androgen receptor (AR) mRNA/protein or PSA mRNA/protein, and comprise a heterozygous P223L/V274F p53 expression pattern.

In any embodiment, the cells of the preparation do not express endogenous KLK2, i.e., the cells only express the kallikrein-2 fusion protein as described herein. In any embodiment, the cells of the preparation express endogenous KLK2 and express the kallikrein-2 fusion protein as described herein.

A further aspect of the present disclosure is directed to a non-human animal comprising cells that express, on their surface, a recombinant kallikrein-2 fusion protein, where the recombinant fusion protein includes a kallikrein-2 polypeptide sequence, a portion of a glycosylphophatidylinositol (GPI) attachment sequence linked to the C-terminus of the kallikrein-2 polypeptide sequence, and GPI anchor domain coupled to the GPI attachment sequence portion.

In one embodiment, the cells expressing the recombinant kallikrein-2 fusion protein are transplanted into the non-human animal. In one embodiment, cells expressing the recombinant kallikrein-2 fusion protein are transplanted into rodent. In one embodiment, cells expressing the recombinant kallikrein-2 fusion protein are transplanted into a mouse. In one embodiment, human cells expressing the recombinant kallikrein-2 fusion protein are transplanted into an immunocompromised rodent, e.g., an immunocompromised mouse. In one embodiment, mouse cells expressing the recombinant kallikrein-2 fusion protein are transplanted into a syngeneic mouse.

In another embodiment, the recombinant nucleic acid construct encoding a kallikrein-2 fusion protein is stably integrated into the genome of the non-human animal to produce a transgenic non-human animal capable of expressing the kallikrein-2 fusion protein on the surface of all or certain subtypes of its cells as described herein.

The recombinant nucleic acid construct encoding the kallikrein-2 fusion protein as described supra can be integrated into the genome of a non-human animal by any standard method well known to those skilled in the art. Any of a variety of techniques known in the art can be used to introduce the transgene into an animal to produce the founder line of transgenic animals (see e.g., Hogan et al., Manipulating the Mouse Embryo: A Laboratory Manual (Cold Spring Harbor Laboratory, 1986); Hogan et al., Manipulating the Mouse Embryo: A Laboratory Manual (Cold Spring Harbor Laboratory, 1994), and U.S. Pat. No. 5,602,299 to Lazzarini; U.S. Pat. No. 5,175,384 to Krimpenfort; U.S. Pat. No. 6,066,778 to Ginsburg; and U.S. Pat. No. 6,037,521 to Sato et al, which are hereby incorporated by reference in their entirety). Such techniques include, but are not limited to, pronuclear microinjection (U.S. Pat. No. 4,873,191 to Wagner et al., which is hereby incorporated by reference in its entirety); retrovirus mediated gene transfer into germ lines (Van der Putten et al., Proc. Natl. Acad. Sci. USA 82:6148-6152 (1985), which is hereby incorporated by reference in its entirety); gene targeting in embryonic stem cells (Thompson et al., Cell 56:313-321 (1989), which is hereby incorporated by reference in its entirety); electroporation of embryos (Lo et al., Mol. Cell. Biol. 3:1803-1814 (1983), which is hereby incorporated by reference in its entirety); and sperm-mediated gene transfer (Lavitrano et al., Cell 57:717-723 (1989), which is hereby incorporated by reference in its entirety).

In any embodiment, embryonic cells at various developmental stages can be used to introduce transgenes for the production of transgenic animals. Different methods are used depending on the stage of development of the embryonic cell. The zygote is a good target for micro-injection, and methods of microinjecting zygotes are well known to (see U.S. Pat. No. 4,873,191 to Wagner et al., which is hereby incorporated by reference in its entirety). The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985), which is hereby incorporated by reference in its entirety). As a consequence, all cells of the transgenic non-human animal will carry the incorporated transgene.

The transgenic animals of the present invention can also be generated by introduction of the targeting vectors into embryonic stem (ES) cells. ES cells are obtained by culturing pre-implantation embryos in vitro under appropriate conditions (Evans et al., Nature 292:154-156 (1981); Bradley et al., Nature 309:255-258 (1984); Gossler et al., Proc. Natl. Acad. Sci. USA 83:9065-9069 (1986); and Robertson et al., Nature 322:445-448 (1986), which are hereby incorporated by reference in their entirety). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (reviewed in Jaenisch, Science 240:1468-1474 (1988), which is hereby incorporated by reference in its entirety). Prior to the introduction of transfected ES cells into the blastocoel, the transfected ES cells can be subjected to various selection protocols to enrich for ES cells that have integrated the transgene if the transgene provides a means for such selection.

In addition, retroviral infection can also be used to introduce transgenes into a non-human animal. The developing non-human embryo can be cultured in vitro to the blastocyst stage. During this time, the blastomeres can be targets for retroviral infection (Janenich, Proc. Natl. Acad. Sci. USA 73:1260-1264 (1976), which is hereby incorporated by reference in its entirety). The viral vector system used to introduce the transgene is typically a replication-defective retrovirus carrying the transgene (Jahner et al., Proc. Natl. Acad. Sci. USA 82:6927-6931 (1985); Van der Putten et al. Proc. Natl. Acad. Sci. USA 82:6148-6152 (1985)). Transfection is easily and efficiently obtained by culturing the blastomeres on a monolayer of virus-producing cells. Alternatively, infection can be performed at a later stage. Additional means of using retroviruses or retroviral vectors to create transgenic animals known to the art involves the micro-injection of retroviral particles or mitomycin C-treated cells producing retrovirus into the perivitelline space of fertilized eggs or early embryos (WO 90/08832 to Onions, which is hereby incorporated by reference in its entirety).

In any embodiment, the transgenic non-human animals express the kallikrein-2 fusion protein on the surface of all of their cells. In any embodiment, the transgenic non-human animals express the kallikrein-2 fusion protein in some, but not all their cells, i.e., expression of the fusion protein is controlled by a cell specific promoter and/or enhancer elements placed upstream of the transgene. In one embodiment, the transgenic non-human animal expresses the kallikrein-2 fusion protein in only prostate cells. In accordance with this embodiment of the disclosure, a prostate cell specific promoter sequence is operably linked to the recombinant nucleic acid construct encoding the kallikrein-2 fusion protein. Suitable prostate specific promoters include, without limitation, the prostate-specific antigen (PSA) promoter, the probasin promoter, prostate-specific membrane antigen (PSMA), and mouse mammary tumor virus (MMTV LTR) promoter. Expression or cloning constructs suitable for driving transgene expression in a transgenic animal are well known in the art. Other components of the expression construct include a strong polyadenylation site, appropriate restriction endonuclease sites, and introns to ensure the transcript is spliced.

The recombinant nucleic acid construct encoding the kallikrein-2 fusion protein can be inserted into any non-human animal. Preferably, the animal is a rodent, more preferably, the animal is a mouse. Suitable strains of mice commonly used in the generation of transgenic models include, without limitation, CD-i® Nude mice, NU/NU mice, BALB/C Nude mice, BALB/C mice, NIH-III mice, SCID® mice, outbred SCID® mice, SCID Beige mice, C3H mice, C57BL/6 mice, DBA/2 mice, FVB mice, CB17 mice, 129 mice, SJL mice, B6C3F1 mice, BDF1 mice, CDF1 mice, CB6F1 mice, CF-1 mice, Swiss Webster mice, SKH1 mice, PGP mice, and B6SJL mice.

In any embodiment, the recombinant nucleic acid construct encoding the kallikrein-2 fusion protein is introduced into a non-murine mammal, such as sheep, goats, pigs, dogs, cats, monkeys, chimpanzees, hamsters, rabbits, cows, and guinea pigs (see, e.g., Kim et al., “Development of a Positive Method for Male Stem-cell Mediated Gene-transfer in Mouse and Pig,” Mol. Reprod. Dev. 46(4): 515-526 (1997); Houdebine, “The Production of Pharmaceutical Proteins from the Milk of Transgenic Animals,” Reprod. Nutr. Dev. 35(6):609-617 (1995); Petters, “Transgenic Livestock as Genetic Models of Human Disease,” Reprod. Fertil. Dev. 6(5):643-645 (1994); Schnieke et al., “Human Factor IX Transgenic Sheep Produced by Transfer of Nuclei from Transfected Fetal Fibroblasts,” Science 278(5346):2130-2133 (1997); Amoah & Gelaye, “Biotechnology Advances in Goat Reproduction,” J. Animal Science 75(2):578-585 (1997), which are hereby incorporated by reference in their entirety).

The transgenic animals are screened and evaluated to select those animals having a phenotype wherein the kallikrein-2 fusion protein is expressed on all cells or on a subset of cells, e.g., prostate cells specifically. Initial screening can be performed using, for example, Southern blot analysis or PCR techniques to analyze animal cells to verify that integration of the transgene has taken place. The level of mRNA expression of the transgene in the cells of the transgenic animals can also be assessed using techniques which include, but are not limited to, Northern blot analysis of tissue samples obtained from the animal, in situ hybridization analysis, and reverse transcriptase-PCR (rt-PCR). In addition, surface expression of the kallikrein-2 fusion protein can be evaluated by flow cytometry using human-specific anti-kallikrein-2 antibodies as described herein (e.g., antibodies KL2B1, KL2B53, and KL2B30)

Another aspect of the present disclosure is directed to methods of identifying kallikrein-2 targeting therapeutic agents. In any embodiment, a therapeutic kallikrein-2 targeting agent is one that binds to kallikrein-2 to cause a therapeutic endpoint (e.g., induce cell death). In any embodiment, a therapeutic kallikrein-2 targeting agent is one that directly binds to or otherwise interacts with kallikrein-2 to modulate kallikrein-2 expression, activity, or function. In any embodiment, the therapeutic kallikrein-2 targeting agent is one that binds to or otherwise interacts with kallikrein-2 to delivery an active agent to the cell expressing kallikrein-2 on its surface. In any embodiment, a therapeutic kallikrein-2 targeting agent is one that binds to kallikrein-2 and to immune cells (e.g., T lymphocytes, natural killer cells, macrophages, iPSC-derived T cells or iPSC-derived NK cells) simultaneously to mediate killing of the cell expressing kallikrein-2 on its surface by the immune cells.

In accordance with this aspect of the disclosure, the method of identifying kallikrein-2 targeting agents involves providing a preparation of cells as described herein, where cells of the preparation express, on their surface, the kallikrein-2 fusion protein (e.g., a fusion protein comprising a kallikrein-2 polypeptide sequence, a portion of a glycosylphophatidylinositol (GPI) attachment sequence linked to the C-terminus of the kallikrein-2 polypeptide sequence, and GPI anchor domain coupled to the GPI attachment sequence portion). The method further involves administering a candidate kallikrein-2 targeting agent to the preparation of cells and determining whether the candidate agent binds kallikrein-2 or otherwise modifies kallikrein-2 expression, function, or activity based on said administering.

In any embodiment, the method further involves providing a second preparation of cells, where cells of the second preparation have not been modified to express the kallikrein-2 fusion protein as described herein. A comparison of the endpoint utilized to determine whether the candidate agent binds to kallikrein-2 or otherwise modifies kallikrein-2 function, expression, or activity between the cell preparation modified to express the kallikrein-2 fusion protein and the cell preparation not expressing the kallikrein-2 fusion protein (i.e., the control cell preparation) demonstrates the kallikrein-2 antigen specificity of the candidate agent. In any embodiment, the second preparation of cells is isogenic to the cell preparation modified to express the kallikrein-2 fusion protein.

Suitable preparations of cells for use in the methods described herein are described in detail supra. In any embodiment, the preparation of cells is a preparation of cancer cells. In any embodiment, the preparation of cells is a preparation of prostate cancer (PCa) cells.

Alternatively, this method involves providing a non-human animal comprising cells that express one their surface, a recombinant kallikrein-2 fusion protein. As described supra, the kallikrein-2 fusion protein of the non-human animal includes a kallikrein-2 polypeptide sequence, a portion of a glycosylphophatidylinositol (GPI) attachment sequence linked to the C-terminus of the kallikrein-2 polypeptide sequence, and GPI anchor domain coupled to the GPI attachment sequence portion. The method further involves administering a candidate kallikrein-2 targeting therapeutic agent to the non-human animal, and determining whether the candidate agent binds kallikrein-2 based on said administering. Administering the candidate kallikrein-2 therapeutic agent to the non-human animal can be carried out using any suitable means, e.g., by parenteral, topical, oral, intravenous, subcutaneous, peritoneal, intranasal or intratumoral means of administration.

In any embodiment, the method further involves providing a second non-human animal that does not comprise cells modified to express the kallikrein-2 fusion protein as described herein. A comparison of the endpoint utilized to determine whether the candidate agent binds to kallikrein-2 or otherwise modifies kallikrein-2 function, expression, or activity between the non-human animal comprising a cell preparation modified to express the kallikrein-2 fusion protein and non-human animals lacking such modified cells demonstrates the kallikrein-2 antigen specificity of the candidate agent. In any embodiment, the second non-human animal is isogenic to the non-human animal comprising cells modified to express the kallikrein-2 fusion protein.

Suitable non-human animals according to the present disclosure are described in more detail supra.

In accordance with these methods, the candidate agent is any candidate kallikrein-2 targeting therapeutic. Suitable candidate targeting therapeutics include, without limitation, any chemical or pharmaceutical entity (e.g., small molecule kallikrein-2 binding agents), a biological kallikrein-2 binding molecule (e.g., kallikrein-2 binding peptide, anti-kallikrein-2 antibody, antibody fragment, monobody, etc.), kallikrein-2 chimeric antigen receptor (CAR) T or NK cell therapy.

In any embodiment, the candidate kallikrein-2 targeting agent includes a detectable label (e.g., the agent can be directly or indirectly detectable). In some cases, the candidate kallikrein-2 targeting agent is directly labeled (e.g., the agent can include a directly detectable adduct, such as a fluorescent adduct). In some cases, the candidate agent is indirectly labeled (e.g., the agent can include an indirectly detectable adduct, such as biotin).

In any embodiment, determining whether the candidate kallikrein-2 targeting agent binds to or otherwise interacts with the kallikrein-2 fusion protein can be accomplished by measuring the amount of the candidate agent bound to the cell expressing the kallikrein-2 fusion protein. Measuring the amount of the candidate agent bound to the cell expressing the kallikrein-2 fusion protein can provide qualitative or quantitative results. In any embodiment, measuring can be carried out using flow cytometry, ELISA, or any other method that can quantitatively measure the amount of candidate agent present or bound to the cells expressing the kallikrein-2 fusion protein. The amount (level) of the candidate agent bound can be expressed in arbitrary units associated with a particular assay (e.g., fluorescence units, e.g., mean fluorescence intensity (MFI)), or can be expressed as an absolute value with defined units (e.g., number of molecules (e.g., moles), number of protein molecules, concentration of agent, etc.). Additionally, a quantitatively measured amount (level) can be compared to the amount of a reference value to derive a normalized value that represents a normalized measured amount.

In any embodiment determining whether the candidate agent is a kallikrein-2 targeting therapeutic or otherwise interacts with the kallikrein-2 fusion protein can be accomplished by measuring a downstream therapeutic endpoint, e.g., antibody-dependent cellular cytotoxicity or complement-dependent cytotoxicity. Methods of measuring cellular cytotoxicity, cell death, and/or cell viability are well known to those of skill in the art.

The following examples are provided to further describe some of the embodiments disclosed herein. The examples are intended to illustrate, not to limit, the disclosed embodiments. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Example 1—Cell Surface Expression of a Kallikrein-2 Fusion Protein

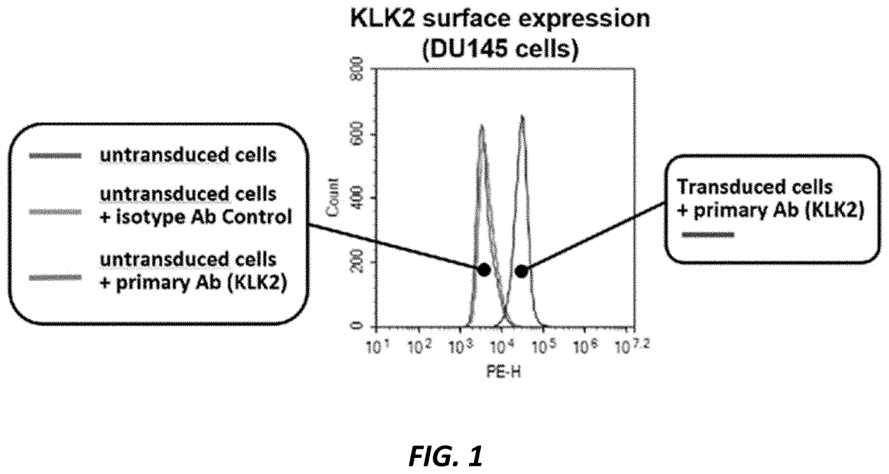

The huKLK2_GPI gene was successfully cloned into pCDH Neo vector at 5′ XbaI and 3′ BamHI restriction sites (SEQ ID NO:7). The scaled up plasmid DNA was sequence confirmed. Lentivirus was produced in HEK293TN cells and transduced into DU145 cells in complete media (EMEM+10% FBS+1×MEM-NEAA+1×Sodium Pyruvate) containing TransDux™. Cells transduced with the KLK2-GPI gene were selected in 1 mg/ml Geneticin and analyzed for KLK2 surface expression by flow cytometry. Surface expression of KLK2 was assessed using the KL2B1 antibody conjugated to phycoerythrin (Janssen). Surface expression was also assessed by a KLK2 antibody procured from R&D Systems (human kallikrein 2 antibody; clone 426723; R&D Systems; Cat #MAB4104) followed by a secondary goat anti mouse detection antibody conjugated to phycoerythrin (Southern Biotech; cat. #1030-09). Expression of KLK2-GPI was detected on the cell surface of transduced cells by both the Janssen antibody (FIG. 1 and Table 3) and the R&D Systems antibody.

TABLE 3
Fluorescence Intensity in DU145 Cells Stained with KLK2 Antibody

| Sample | Mean | Median |
|---|---|---|
| Untransduced DU145 cells | 4,646 | 3,832 |
| Untransduced DU145 cells + isotype control Ab | 5,342 | 4,437 |
| Untransduced DU145 cells + KLK-PE Ab | 5,329 | 4,456 |
| Transduced DU145 cells + KLK2-PE Ab (Viral dilution: 1:2) | 32,745 | 31,139 |
| Transduced DU145 cells + KLK2-PE Ab (Viral dilution: 1:5) | 33,432 | 31,836 |

### Example 2—Assessment of DU145/KLK2_GPI and PC3/KLK2_GPI Cell Lines

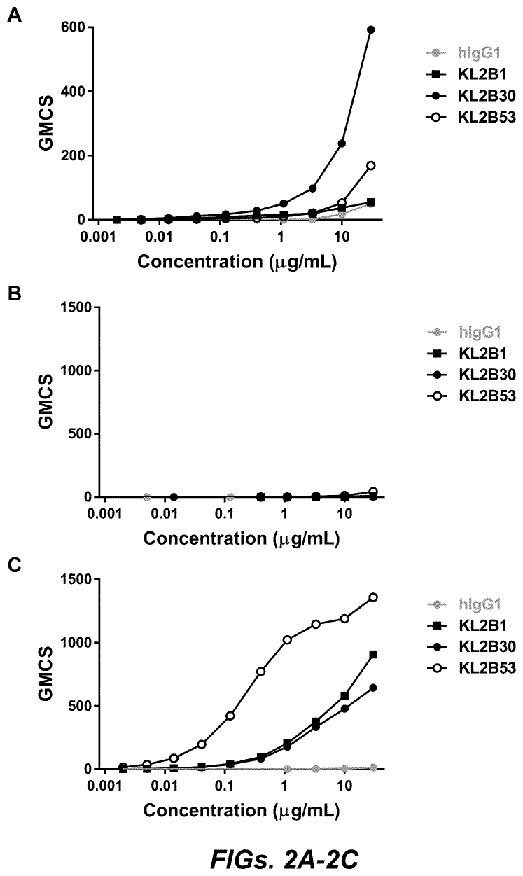

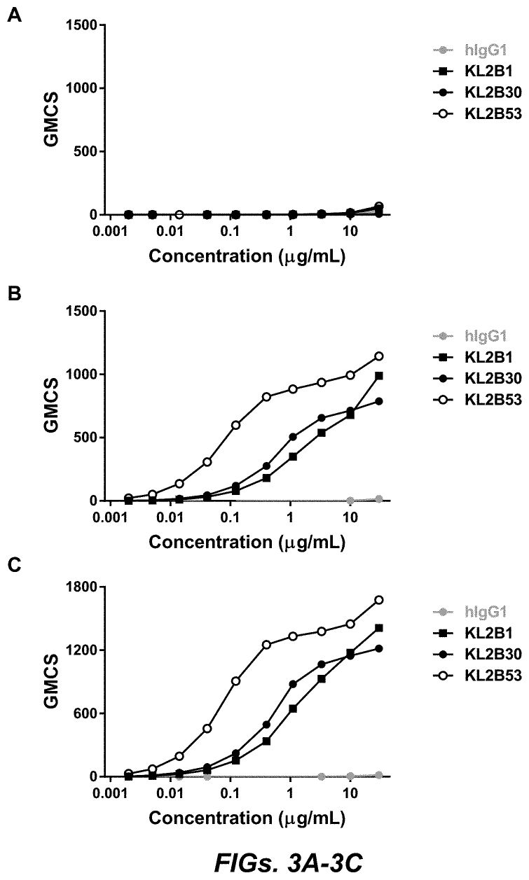

GPI-anchored KLK2 was engineered into DU145 or PC3 prostate tumor cell lines as described in Example 1 above. KLK2 cell surface expression was confirmed by flow cytometry using aKLK2-specific antibodies (Abs) (clones KL2B1, KL2B30 or KL2B53) (FIGS. 2A-2C). KL2B1, KL2B30, and KL2B53 recognize different epitopes on KLK2 protein and show different binding affinities to VCaP cells (FIG. 2A). In contrast, these Abs did not recognize parental DU145 or PC3 tumor cells which did not express KLK2 (FIG. 2B and FIG. 3A). Expression of GPI-anchored KLK2 led to binding of these Abs to engineered DU145/KLK2_GPI and PC3/KLK2_GPI tumor cells (FIG. 2C and FIG. 3B). Co-expression of KLK2_GPI and PSMA was also possible, creating cell lines that are positive for both KLK2 and PSMA which are useful for the validation of dual targeting therapeutic strategies (FIG. 3C).

Three different therapeutic modalities were used to assess the DU145/KLK2_GPI and PC3/KLK2_GPI cell lines—(1) aKLK2 antibody-dependent cell-mediated cytotoxicity (ADCC), (2) KLK2×CD3 bispecific antibodies, and (3) aKLK2 CAR-T cells.

aKLK2-Mediated ADCC Assays

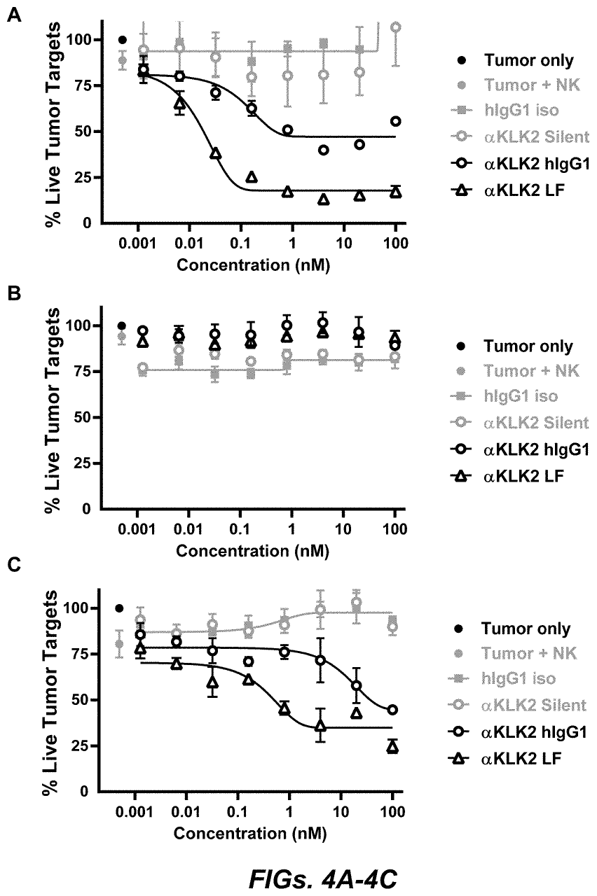

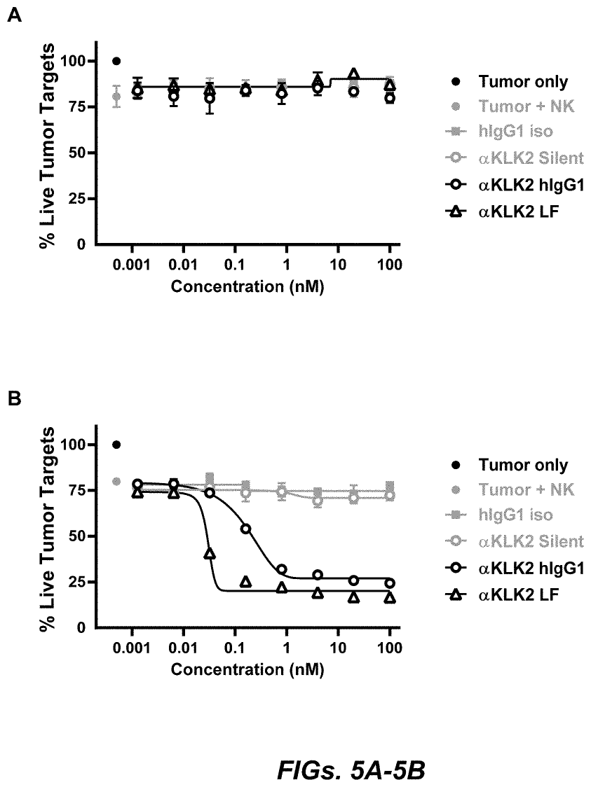

For aKLK2-mediated ADCC assays, healthy donor peripheral blood NK cells (PB-NK) were co-cultured with VCaP, DU145, or PC3 prostate tumor cells with or without KLK2 expression (FIGS. 4A-4C and FIGS. 5A-5B). VCaP tumor cell line is the only tumor line that expresses endogenous KLK2 on the cell surface. These tumor cells can be lysed by PB-NK in the presence of aKLK2 antibodies on either hIgG1 Fc or low fucosylated Fc (LF) (FIG. 4A). Isotype control (hIgG1 iso) or aKLK2 on a silent Fc (aKLK2 Silent) failed to mediate ADCC against VCaP cells. Results in FIGS. 4A-4C further demonstrate that aKLK2 on hIgG1 Fc or LF mediated ADCC against DU145/KLK2_GPI in a dose-dependent manner, but not against DU145 parental cells which do not express KLK2. The low fucosylated aKLK2 (aKLK2 LF) Ab was more potent than the same antibody on wildtype human IgG1 Fc (aKLK2 hIgG1) against VCaP or DU145/KLK2_GPI, indicating that LF Ab enhances ADCC relative to normal fucose hIgG1. Isotype control (hIgG1 iso) or aKLK2 on a silent Fc (aKLK2 Silent) failed to mediate ADCC against DU145/KLK2_GPI tumor cells. Similar results were observed in PC3/KLK2_GPI prostate tumor cells (FIGS. 5A-5B). These findings demonstrate KLK2 antigen-directed killing of tumor targets and the utility of isogenic cell line pairs. Since multiple attempts to knock out KLK2 in VCaP tumor cells have failed, the use of the new isogenic cell line pairs is critical to demonstrate KLK2 antigen-specific response by KLK2-targeting therapeutics.

### KLK2×CD3 Bispecific Ab-Mediated Killing Assays

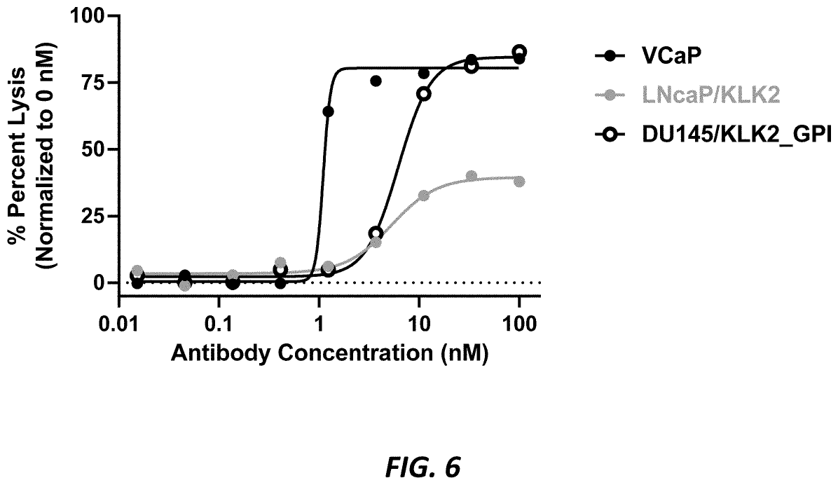

For KLK2×CD3 bispecific Ab-mediated killing assays, healthy donor peripheral blood T cells were co-cultured with VCaP, LnCap/KLK2, or DU145/KLK2_GPI tumor cells (FIG. 6). The KLK2×CD3 bispecific Ab induced dose-dependent lysis of all three target cells with the highest sensitivity against the endogenously expressed VCaP cells. Killing against the DU145/KLK2_GPI tumor cells was not as potent as VCaP, but the maximal levels of killing were similar between the two cell lines, indicating the KLK2 anchored via GPI was recognized by the bispecific Ab. Maximal killing against LnCap/KLK2 is significantly lower, potentially due to the relatively low expression level of KLK2 displayed on LnCap compared to VCaP and DU145/KLK2_GPI. KLK2 expression in the LnCap/KLK2 cell line is not GPI anchor. This further demonstrates that GPI-anchored KLK2 is an useful tool to express KLK2 on cell surface at a high level.

### CAR-T Functionality Assessment

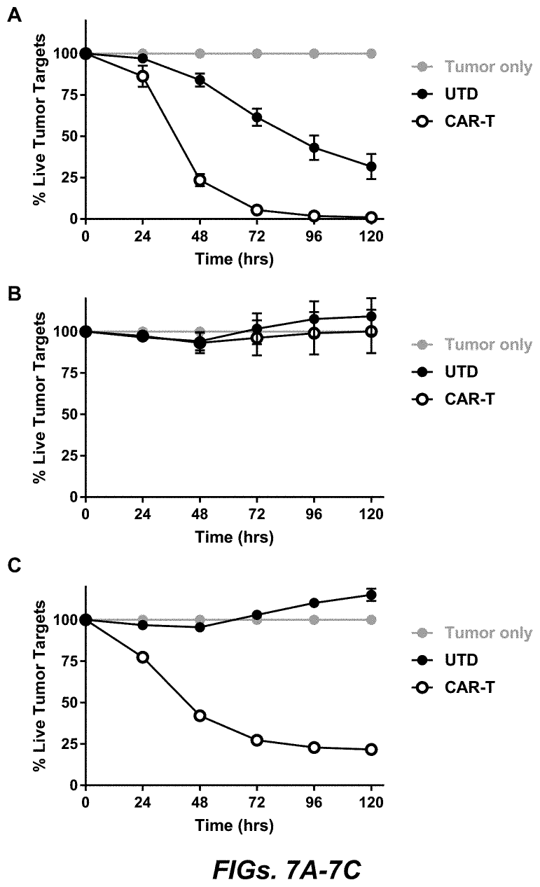

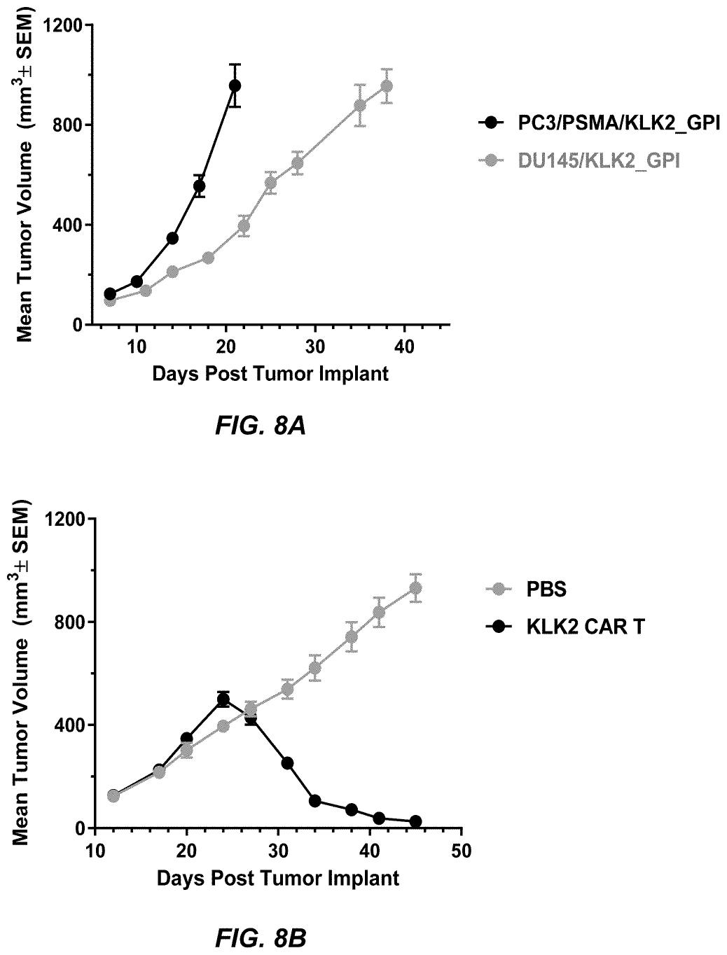

For CAR-T functionality assessment, healthy donor T cells were transduced with KLK2 CAR and co-cultured with VCaP, parental DU145, or DU145/KLK2_GPI (FIGS. 7A-7C). Although untransduced T cells (UTD) killed VCaP cells at a modest level due to allogeneic recognition, KLK2 CAR-T killed VCaP cells more effectively than untransduced T cells, demonstrating CAR-mediated cytotoxicity (FIG. 7A). In addition, KLK2 CAR-T demonstrated KLK2-specific cytolysis against DU145/KLK2_GPI, but not parental DU145 tumor cells (FIG. 7B and FIG. 7C). Again, these findings indicate that GPI-anchored KLK2-expressing prostate cell lines are important tools to demonstrate KLK2 specificity. It also further underscores the importance of isogenic tumor cells to demonstrate KLK2 antigen-specific response by KLK2-targeting therapeutics.

### DU145+KLK2 Cells can be Used to Screen CAR Designs

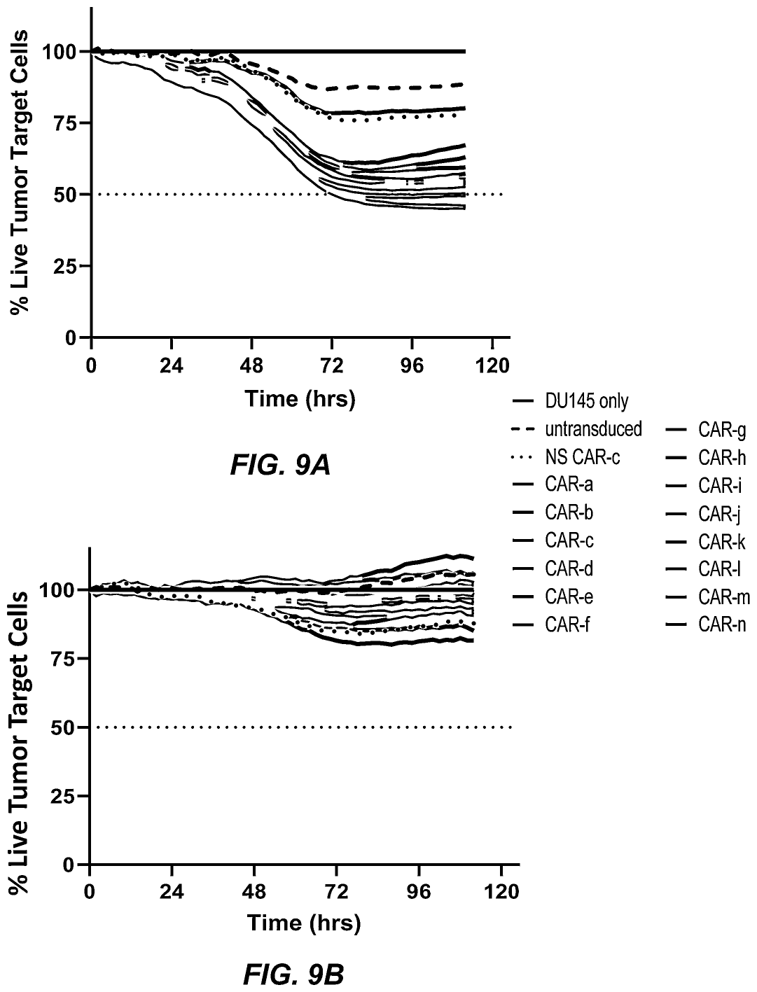

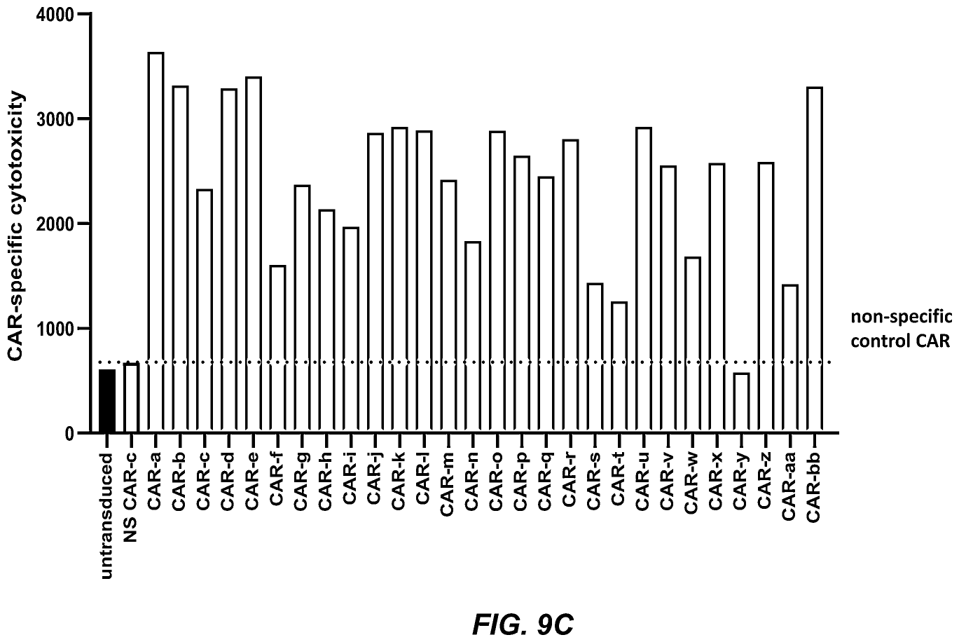

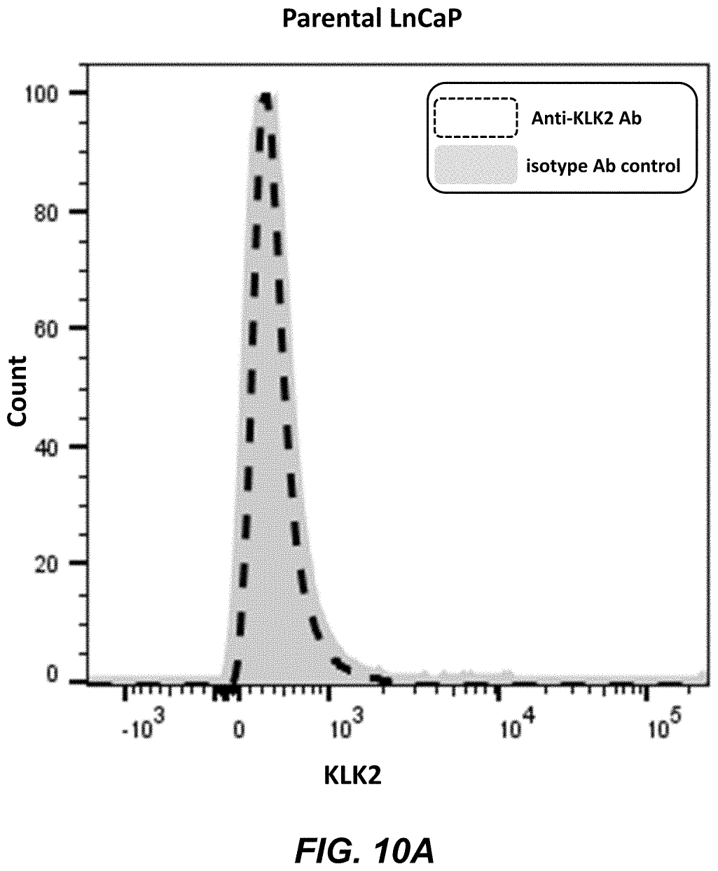

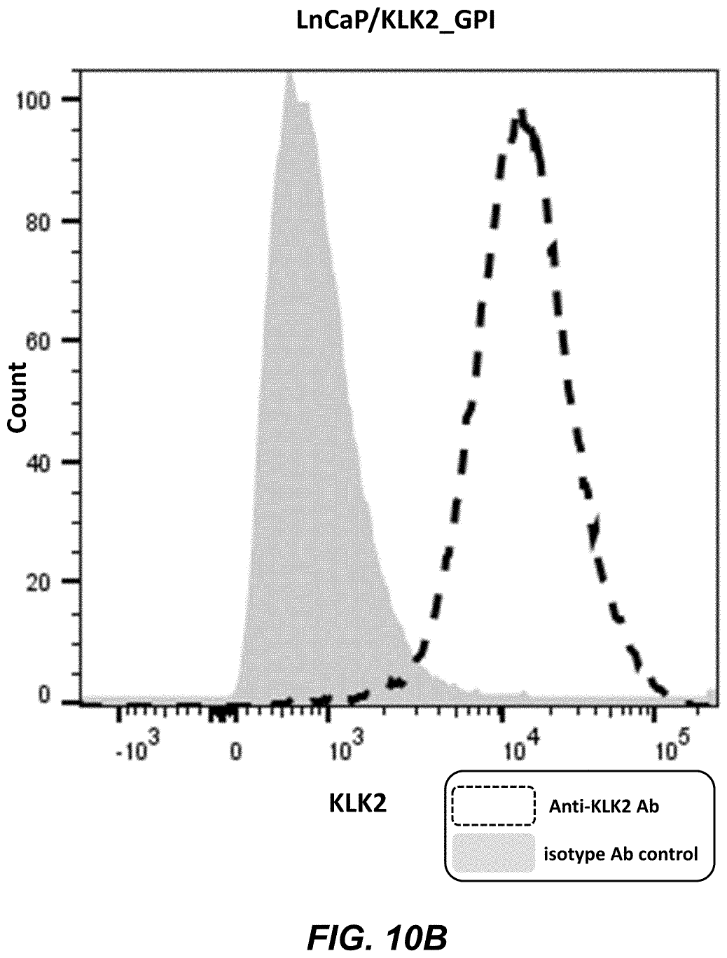

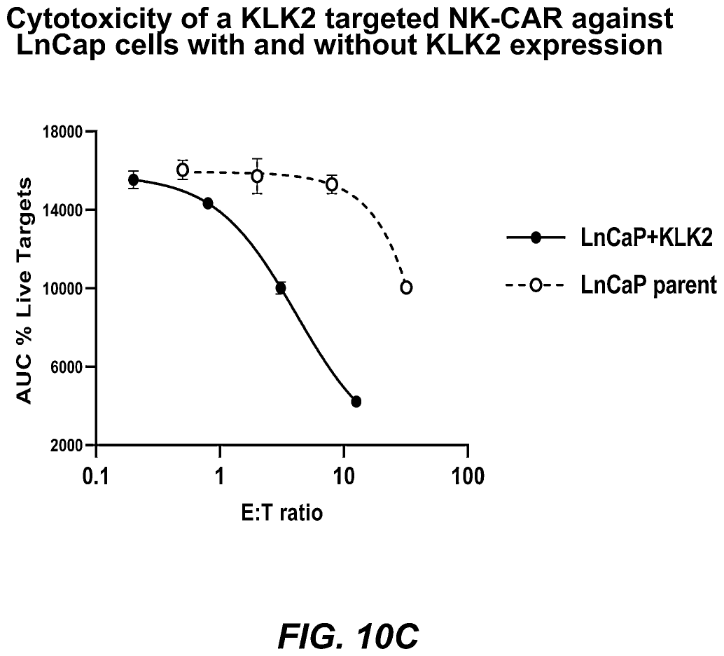

NK-101 cells that stably express each design were sorted with an antibody to the binding domain of the CAR such that the population of CAR expressing cells ranged from 86-99% pure. These effector NK-101+CAR cells were co-cultured at an E:T ratio of 0.5:1 with DU145 target tumor cells that either express (FIG. 9A) or do not express (FIG. 9B) KLK2. The number of live tumor target cells remaining in each well were counted every 2 hours for 5 days using IncuCyte and normalized to tumor only wells to generate % live tumor target cells. To determine the amount of innate killing not mediated by the CAR, DU145 parent cells that do not express KLK2 were also tested. CAR-specific cytotoxicity was determined by the formula: CAR-specific cytotoxicity=(AUC_{DU145 parent})−(AUC_{DU145+KLK2}) and plotted as in (FIG. 9C). Controls included untransduced NK-101 cells and also NK-101 cells expressing a non-specific CAR (NS CAR-c) that did not bind to KLK2 or anything else on the target cells.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A recombinant nucleic acid construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

2. The construct of claim 1, wherein kallikrein-2 is human kallikrein-2.

3. The construct of claim 1, wherein the first nucleotide sequence encodes kallikrein-2 comprising the amino acid sequence of SEQ ID NO: 4 or a fragment thereof.

4. The construct of claim 1, wherein the first nucleotide sequence encoding kallikrein-2 comprises the nucleotide sequence of SEQ ID NO: 1 or a fragment thereof.

5. The construct of claim 1, wherein the GPI attachment sequence is derived from alkaline phosphatase.

6. The construct of claim 5, wherein the GPI attachment sequence is derived from human placental alkaline phosphatase.

7. The construct of claim 5, wherein the second nucleotide sequence encodes a GPI attachment sequence comprising the amino acid sequence of SEQ ID NO: 5 or a fragment thereof.

8. The construct of claim 7, wherein the second nucleotide sequence encoding the GPI attachment sequence comprises the nucleotide sequence of SEQ ID NO: 2.

9. The construct of claim 1, wherein the first and second nucleotide sequences of the construct encode a kallikrein-2 fusion protein comprising the amino acid sequence of SEQ ID NO: 6 or SEQ ID NO: 7.

10. The construct of claim 1, wherein the first and second nucleotide sequences of the construct comprise a nucleotide sequence having at least 90% sequence identity to the nucleotide sequence of SEQ ID NO: 3.

11. The construct of claim 10, wherein the construct comprises the nucleotide sequence of SEQ ID NO: 3.

12. The construct of claim 1 further comprising:
a promoter nucleotide sequence positioned 5′ to the first nucleotide sequence encoding kallikrein-2.

13. The construct of claim 12, wherein the promoter nucleotide sequence is a mammalian promoter sequence.

14. The construct of claim 13, where the promoter nucleotide sequence is an EF1α promoter nucleotide sequence

15. A vector comprising the construct of claim 1.

16. The vector of claim 15, wherein said vector is a viral vector.

17. The vector of claim 16, wherein said viral vector is selected from the group consisting of an adenoviral vector, an adeno-associated viral vector, a lentivirus vector, a vaccinia vector, a retroviral vector, and a herpes simplex viral vector.

18. A cell comprising the recombinant construct of claim 1. or the vector of claim 15.

19. A kallikrein-2 fusion protein encoded by the construct of claim 1.

20. The kallikrein-2 fusion protein of claim 19, wherein said fusion protein comprises an amino acid sequence having at least 90% sequence identity to the amino acid sequence of SEQ ID NO: 6.

21. A preparation of cells, wherein cells of the preparation are modified to express, on their surface, a recombinant kallikrein-2 fusion protein, said fusion protein comprising:
a kallikrein-2 polypeptide sequence;
a portion of a glycosylphophatidylinositol (GPI) attachment sequence linked to the kallikrein-2 polypeptide sequence at its C-terminus; and
a GPI anchor domain coupled to the portion of the GPI attachment sequence.

22. The preparation of claim 21, wherein the kallikrein-2 polypeptide sequence is a human kallikrein-2 polypeptide sequence.

23. The preparation of claim 21, wherein the kallikrein-2 polypeptide sequence comprises an amino acid sequence having at least 90% sequence identity to the amino acid sequence of SEQ ID NO: 4 or a fragment thereof.

24. The preparation of claim 23, wherein the kallikrein-2 polypeptide sequence comprises an amino acid sequence of SEQ ID NO: 4 or a fragment thereof.

25. The preparation of claim 21, wherein the portion of the GPI attachment sequence is derived from alkaline phosphatase.

26. The preparation of claim 21, wherein the portion of the GPI attachment sequence is derived from human placental alkaline phosphatase.

27. The preparation of claim 21 wherein the portion of the GPI attachment sequence comprises a portion of the amino acid sequence of SEQ ID NO: 5.

28. The preparation of claim 21, wherein the kallikrein-2 fusion protein comprises an amino acid sequence having at least 90% sequence identity to the amino acid sequence of SEQ ID NO: 7.

29. The preparation of claim 28, wherein the kallikrein-2 fusion protein comprises the amino acid sequence of SEQ ID NO: 7.

30. The preparation of claim 21, wherein cells of the preparation express the kallikrein-2 fusion protein from the recombinant construct of claim 1.

31. The preparation of claim 30, wherein cells of the preparation comprise the recombinant construct stably integrated into their genome.

32. The preparation of claim 21, wherein the cells of the preparation are mammalian cells.

33. The preparation of claim 21, wherein cells of the preparation are human cells.

34. The preparation of claim 21 wherein cells of the preparation are rodent cells.

35. The preparation of claim 34, wherein the rodent cells are mouse cells.

36. The preparation of claim 21, wherein the cells are prostate cells.

37. The preparation of claim 36, where the prostate cells are prostate cancer cells.

38. The preparation of claim 21, wherein cells of the preparation do not express endogenous kallikrein-2.

39. The preparation of claim 21, wherein the preparation of cells is a cell line.

40. A non-human animal comprising the preparation of cells of claim 21.

41. A non-human animal comprising cells expressing, at their surface, a recombinant kallikrein-2 fusion protein, said fusion protein comprising:
a kallikrein-2 polypeptide sequence;
a portion of a glycosylphophatidylinositol (GPI) attachment sequence linked to the C-terminus of the kallikrein-2 polypeptide sequence; and
a GPI anchor domain coupled to the portion of the GPI attachment sequence.

42. The non-human animal of claim 41, wherein cells of the non-human animal are transduced with the recombinant construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

43. The non-human animal of claim 41, wherein the recombinant fusion protein is stably integrated into the non-human animal's genome.

44. The non-human animal of claim 41, wherein said non-human animal is a rodent.

45. The non-human animal of claim 44, wherein the rodent is a mouse.

46. A method of identifying an agent that binds kallikrein-2, said method comprising:
providing the preparation of cells of claim 21;
administering a candidate agent to the preparation of cells; and
determining whether the candidate agent binds kallikrein-2 based on said administering.

47. A method of identifying an agent that binds kallikrein-2, said method comprising:
providing the non-human animal of claim 40;
administering a candidate agent to the non-human animal; and
determining whether the candidate agent binds kallikrein-2 based on said administering.

48. The method of claim 46, wherein the preparation of cells is a preparation of cancer cells.

49. The method of claim 48, wherein the preparation of cells is a preparation of prostate cancer cells.

50. The method of claim 46, wherein the candidate agent is a candidate kallikrein-2 inhibitor.

51. The method of claim 46, wherein the candidate agent is an anti-kallikrein-2 antibody.

52. The method of claim 46, wherein the candidate agent is a kallikrein-2 chimeric antigen receptor (CAR).

53. A cell comprising a vector comprising a recombinant nucleic acid construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-a.

54. A cell comprising a vector comprising a recombinant nucleic acid construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

55. The preparation of claim 21, wherein cells of the preparation express the kallikrein-2 fusion protein from the vector comprising a recombinant nucleic acid construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.

56. The non-human animal of claim 41, wherein cells of the non-human animal are transduced with the recombinant construct comprising a vector comprising a recombinant nucleic acid construct encoding a kallikrein-2 fusion protein, said construct comprising:
a first nucleotide sequence encoding kallikrein-2 (KLK2) or a fragment thereof, and
a second nucleotide sequence encoding a glycosylphophatidylinositol (GPI) attachment sequence, wherein said second nucleotide sequence encoding the GPI attachment sequence is positioned 3′ to the first nucleotide sequence encoding kallikrein-2.


SEQUENCE LISTING (26 sequences)

SEQ ID NO: 1 - DNA, 789 bp, Artificial Sequence
  Description of Artificial Sequence: Synthetic polynucleotide
atgtgggacctggttctctccatcgccttgtctgtggggtgcactggtgccgtgcccctcatccagtctcggatcgtggggggctgggagtgcgagaagcacagccagccttggcaagtggcagtgtactcccacggttgggcgcactgcggtggcgtgctggtgcacccacaatgggtgctcaccgcggcccactgtctgaagaagaattcacaagtctggctgggacgccataacctgttcgaacctgaagatactgggcagcgcgtgccggtgtcccattccttccctcacccattgtacaacatgtcgctgctgaagcaccagtctttgaggcctgatgaggacagctcccatgacctcatgctgcttagactctcggaacccgcaaagattaccgacgtcgtgaaagtgcttggactgccgacgcaggaacccgccttggggactacctgttatgcttccggctggggatccatcgagcccgaagaattcctgcggccgcgcagcctgcagtgcgtgtccctccatctgctgtcaaacgatatgtgcgccagagcctactccgaaaaggtcaccgagtttatgctgtgcgccggactgtggaccgggggaaaggacacttgcggcggagacagcggcggccccctggtctgcaacggcgtgctgcagggaattacctcgtggggtccagagccgtgtgcgctgcctgaaaagcccgccgtgtacactaaggtcgtgcactaccggaagtggatcaaggacaccatcgccgcgaacccggaattc

SEQ ID NO: 2 - DNA, 96 bp, Artificial Sequence
  Description of Artificial Sequence: Synthetic oligonucleotide
accactgatgctgcccatcctggaaggtctgtggtgcctgccttgctgcctctgctggctggcactctgctgctgctggagactgccactgctccc

SEQ ID NO: 3 - DNA, 891 bp, Artificial Sequence
  Description of Artificial Sequence: Synthetic polynucleotide
atgtgggacctggttctctccatcgccttgtctgtggggtgcactggtgccgtgcccctcatccagtctcggatcgtggggggctgggagtgcgagaagcacagccagccttggcaagtggcagtgtactcccacggttgggcgcactgcggtggcgtgctggtgcacccacaatgggtgctcaccgcggcccactgtctgaagaagaattcacaagtctggctgggacgccataacctgttcgaacctgaagatactgggcagcgcgtgccggtgtcccattccttccctcacccattgtacaacatgtcgctgctgaagcaccagtctttgaggcctgatgaggacagctcccatgacctcatgctgcttagactctcggaacccgcaaagattaccgacgtcgtgaaagtgcttggactgccgacgcaggaacccgccttggggactacctgttatgcttccggctggggatccatcgagcccgaagaattcctgcggccgcgcagcctgcagtgcgtgtccctccatctgctgtcaaacgatatgtgcgccagagcctactccgaaaaggtcaccgagtttatgctgtgcgccggactgtggaccgggggaaaggacacttgcggcggagacagcggcggccccctggtctgcaacggcgtgctgcagggaattacctcgtggggtccagagccgtgtgcgctgcctgaaaagcccgccgtgtacactaaggtcgtgcactaccggaagtggatcaaggacaccatcgccgcgaacccggaattcaccactgatgctgcccatcctggaaggtctgtggtgcctgccttgctgcctctgctggctggcactctgctgctgctggagactgccactgctccctaatga

SEQ ID NO: 4 - protein, 263 aa, Artificial Sequence
  Description of Artificial Sequence: Synthetic polypeptide
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANPEF

SEQ ID NO: 5 - protein, 32 aa, Artificial Sequence
  Description of Artificial Sequence: Synthetic polypeptide
TTDAAHPGRSVVPALLPLLAGTLLLLETATAP

SEQ ID NO: 6 - protein, 295 aa, Artificial Sequence
  Description of Artificial Sequence: Synthetic polypeptide
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANPEFTTDAAHPGRSVVPALLPLLAGTLLLLETATAP

SEQ ID NO: 7 - DNA, 8724 bp, Artificial Sequence
  Description of Artificial Sequence: Synthetic polynucleotide
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaatttgactagcggaggctagagggagagagatgggtgcgagagcgtcagtattaagcgggggagaataagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaatttttcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatgtgggacctggttctctccatcgccttgtctgtggggtgcactggtgccgtgcccctcatccagtctcggatcgtggggggctgggagtgcgagaagcacagccagccttggcaagtggcagtgtactcccacggttgggcgcactgcggtggcgtgctggtgcacccacaatgggtgctcaccgcggcccactgtctgaagaagaattcacaagtctggctgggacgccataacctgttcgaacctgaagatactgggcagcgcgtgccggtgtcccattccttccctcacccattgtacaacatgtcgctgctgaagcaccagtctttgaggcctgatgaggacagctcccatgacctcatgctgcttagactctcggaacccgcaaagattaccgacgtcgtgaaagtgcttggactgccgacgcaggaacccgccttggggactacctgttatgcttccggctggggatccatcgagcccgaagaattcctgcggccgcgcagcctgcagtgcgtgtccctccatctgctgtcaaacgatatgtgcgccagagcctactccgaaaaggtcaccgagtttatgctgtgcgccggactgtggaccgggggaaaggacacttgcggcggagacagcggcggccccctggtctgcaacggcgtgctgcagggaattacctcgtggggtccagagccgtgtgcgctgcctgaaaagcccgccgtgtacactaaggtcgtgcactaccggaagtggatcaaggacaccatcgccgcgaacccggaattcaccactgatgctgcccatcctggaaggtctgtggtgcctgccttgctgcctctgctggctggcactctgctgctgctggagactgccactgctccctaatgaggatccgcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggcgtccggaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccgcgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 8 - skipped

SEQ ID NO: 9 - protein, 42 aa, Artificial Sequence
  Description of Artificial Sequence: Synthetic polypeptide
TACDLAPPAGTTDAAHPGRSVVPALLPLLAGTLLLLETATAP

SEQ ID NO: 10 - protein, 41 aa, Artificial Sequence
  Description of Artificial Sequence: Synthetic polypeptide
HETTPNKGSGTTSGTTRLLSGHTCFTLTGLLGTLVTMGLLT

SEQ ID NO: 11 - protein, 35 aa, Artificial Sequence
  Description of Artificial Sequence: Synthetic polypeptide
PGESGTSGWRGGDTPSPLCLLLLLLLLILRLLRIL

SEQ ID NO: 12 - protein, 35 aa, Artificial Sequence
  Description of Artificial Sequence: Synthetic polypeptide
ESAEPSRGENAAQTPRIPSRLLAILLFLLAMLLTL

SEQ ID NO: 13 - protein, 29 aa, Artificial Sequence
  Description of Artificial Sequence: Synthetic peptide
YAAAMSGAGPWAAWPFLLSLALMLLWLLS

SEQ ID NO: 14 - protein, 35 aa, Artificial Sequence
  Description of Artificial Sequence: Synthetic polypeptide
PEVRVLHSIGHSAAPRLFPLAWTVLLLPLLLLQTP

SEQ ID NO: 15 - protein, 29 aa, Artificial Sequence
  Description of Artificial Sequence: Synthetic peptide
SVRGINGSISLAVPLWLLAASLLCLLSKC

SEQ ID NO: 16 - protein, 31 aa, Artificial Sequence
  Description of Artificial Sequence: Synthetic polypeptide
DSEGSGALPSLTCSLTPLGLALVLWTVLGPC

SEQ ID NO: 17 - protein, 32 aa, Artificial Sequence
  Description of Artificial Sequence: Synthetic polypeptide
VSQVKISGAPTLSPSLLGLLLPAFGILVYLEF

SEQ ID NO: 18 - protein, 35 aa, Artificial Sequence
  Description of Artificial Sequence: Synthetic polypeptide
QVPKLEKSISGTSPKREHLPLAVGIAFFLMTFLAS

SEQ ID NO: 19 - protein, 35 aa, Artificial Sequence
  Description of Artificial Sequence: Synthetic polypeptide
EAPEPIFTSNNSCSSPGGCRLFLSTIPVLWTLLGS

SEQ ID NO: 20 - protein, 30 aa, Artificial Sequence
  Description of Artificial Sequence: Synthetic polypeptide
TNATTKAAGGALQSTASLFVVSLSLLHLYS

SEQ ID NO: 21 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 22 - DNA, 589 bp, Artificial Sequence
  Description of Artificial Sequence: Synthetic polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 23 - DNA, 1718 bp, Artificial Sequence
  Description of Artificial Sequence: Synthetic polynucleotide
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 24 - DNA, 511 bp, Artificial Sequence
  Description of Artificial Sequence: Synthetic polynucleotide
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 25 - DNA, 344 bp, Artificial Sequence
  Description of Artificial Sequence: Synthetic polynucleotide
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 26 - DNA, 1177 bp, Artificial Sequence
  Description of Artificial Sequence: Synthetic polynucleotide
ggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa